(19) United States
(12) Patent Application Publication
(10) Pub. No.: US 2024/0425465 A1
(43) Pub. Date: Dec. 26, 2024

(54) PYRIDAZINEDIONE-BASED HETEROBICYCLIC COVALENT LINKERS AND METHODS AND APPLICATIONS THEREOF

(71) Applicant: SyntaBio LLC, San Diego, CA (US)

(72) Inventors: Hui Liu, Chula Vista, CA (US); Patrick Li, Chula Vista, CA (US)

(21) Appl. No.: 18/704,873

(22) PCT Filed: Oct. 21, 2022
(86) PCT No.: PCT/US2022/078563

§ 371 Date: Apr. 25, 2024

Related U.S. Application Data
(60) Provisional application No. 63/271,692, filed on Oct. 25, 2021.

(51) Int. Cl. C07D 243/02; A61K 31/5025; A61K 31/551; C07D 487/04; C07D 519/00

(52) U.S. Cl. CPC C07D 243/02; A61K 31/5025; A61K 31/551; C07D 487/04; C07D 519/00

(57) ABSTRACT

Pyridazinedione-based heterobicyclic covalent linkers useful for formation of protein-drug conjugates, protein-protein conjugates, or the like, preparation methods thereof, and applications of the linkers in formation of various protein conjugates are disclosed. The present disclosure provides a new class of linkers to overcome the limitations of the existing pyridazinedione-based linkers. These linkers can be synthesized from commercially available starting materials in 1-3 steps and are highly reactive toward the thiols derived from the reduction of disulfide bridges in proteins.

### CROSS-REFERENCE TO RELATED APPLICATION

This application claims priority under 35 U.S.C. § 119(e) to U.S. Provisional Patent Application No. 63/271,692, filed on Oct. 25, 2021, the disclosure of which is incorporated herein by reference in its entirety.

### FIELD OF THE DISCLOSURE

This application relates to heterobicyclic covalent linkers useful for formation of protein-drug conjugates, and methods and applications thereof.

### BACKGROUND OF THE DISCLOSURE

Previous approaches for the preparation of antibody-drug conjugates (ADCs) have used methods that typically result in heterogeneous products containing a mixture of species with different drug-to-antibody ratios (DAR) that could lead to different pharmacokinetic profiles and consequently variable efficacy in vivo. For example, IgG proteins contain four solvent-accessible interchain disulfide bridges in the protein hinge region, and their reduction creates eight reactive thiols. Conjugation to these reduced species results in heterogeneous mixtures of conjugates with DAR loadings of 2-4 drugs per antibody. The cleavage and exchange of the disulfide bridges in these heterogeneous ADCs can result in adverse pharmacokinetics and reduce the metabolic stability of IgG antibodies in plasma (Mauricio Morais et al., Drug Discovery Today: Technologies, 2018, 30, 91). Additionally, some reduced thiol groups that do not participate in bioconjugate reactions can undergo oxidative intramolecular reactions with other thiols, often producing disulfide scrambling products that disrupt the structure and function of the protein in ADCs. The issues of uncontrolled conjugation are particularly problematic for therapeutic applications, where nonideal drug loading and heterogeneous mixtures of bioconjugates can lead to poor pharmacokinetic properties and a narrow therapeutic window (Peter A. Szijj et al., Drug Discovery Today: Technologies 2018, 30, 27).

Cysteine residues have been explored as a route to site-selectively modify proteins in ADC design, and many successfully approved therapeutics have utilized cysteine directed conjugation reagents (Peter A. Szijj et al., Drug Discovery Today: Technologies 2018, 30, 27). Cysteine is the residue of choice over lysine in modifying proteins because of its low abundance (1.9% for cysteine and 5.9% for lysine) and the high nucleophilicity of its sulfhydryl side chain (pKa=8.0 for cysteine and 10.5 for lysine), which could be translated to high selectivity and less byproducts. Among the methods developed, including maleimide, pyridyldithiopropionate, methylsulfonyl phenyloxadiazole, monobromo maleimide, and carbonylacrylic derivatives, the most used strategy for cysteine modification on biomolecules utilizes maleimide reagents (Mauricio Morais et al., Drug Discovery Today: Technologies, 2018, 30, 91; Peter A. Szijj et al., Drug Discovery Today: Technologies 2018, 30, 27; Seah Ling Kuan et al., Chem. Eur. J. 2016, 22, 17112).

Maleimide derivatives have been widely used to incorporate cysteine thiols into antibody derivatives and proteins. However, maleimide conjugates suffer from instability: the thioether can undergo a retro-Michael reaction, converting back to the starting thiol and maleimide. The maleimide moiety, still attached to its payload, reacts with the low concentrations of endogenous thiol present in blood. In addition, two regioisomers and total four diastereomers could possibly form during the key hydrolysis step, which could result in complicated plasma stabilities and pharmacokinetics (Peter A. Szijj et al., Drug Discovery Today: Technologies 2018, 30, 27; Archie Wall et al., Chem. Sci., 2020, 11, 11455; Vesela Kostova et al., Pharmaceuticals 2021, 14, 442).

In order to preserve structure and function of the protein and create re-bridged covalent bonds which are unreactive towards serum thiols, it is crucial that the cysteine re-bridging reagent can react rapidly with both disulfide-derived reduced thiols and form a plasma-stable conjugate to avoid sulfide scrambling issues in plasma. Various thiol-stable chemical technologies have been applied to modification of disulfide bonds in mAbs and their derivatives. These include bissulfone derivatives, dibromoalkyl oxetane derivatives, trivalent arsenous acid, vinylheteroaryl scaffold divinylpyrimidine and divinyltriazine, monobromo maleimide, dibromopyridazinediones, and disulfide substituted maleimides (Mauricio Morais et al., Drug Discovery Today: Technologies, 2018, 30, 91; Seah Ling Kuan et al., Chem. Eur. J. 2016, 22, 17112).

Despite the broad utilization of maleimides, disubstituted maleimides suffer several limitations which have been widely noted. The hydrolysis generates two regioisomeric maleamic acids that may present different plasma stability and pharmacokinetics (Peter A. Szijj et al., Drug Discovery Today: Technologies 2018, 30, 27; Archie Wall et al., Chem. Sci., 2020, 11, 11455).

In particular, dibromopyridazinediones (diBrPDs) have emerged as a class of disulfide-bridging reagents with great potential because they do not require a hydrolysis step to afford serum stability and are tolerant of/compatible with common mild reducing reagents. In addition, dibromopyridazinediones (diBrPDs) enable site-selective attachment of three functionalities to a protein bearing a single cysteine residue (Calise Bahou et al., Org. Biomol. Chem., 2018, 16, 1359). Modification with diBrPDs creates good levels ofhomogeneity, long term blood plasma stability, and has no detectable effect on the binding capability of the parent antibody. Currently, the diBrPD linker platform has been employed extensively in the generation of ADCs, antibody conjugates, and antibody-directed photosensitizers (Marcos Fernandez et al., Chem. Commun., 2020, 56, 1125).

Although many reagents have been developed for cysteine-specific protein re-bridging modification, few of them allow for multi-functionalization of a single Cys residue and disulfide bridging bioconjugation. Even though the protein derivatives using the diBrPD moiety produced good levels of homogeneity and long-term blood plasma stability, it would bring some regioselectivity issues which may complicate the bioconjugate compounds using this connector (Calise Bahou et al., Org. Biomol. Chem., 2018, 16, 1359). For example, the different N-alkyl groups may lead two regioisomers of thiol-linker products which would be very difficult to separate. If these antibody regioisomers are carried into ADC product, a complicated plasma stability and inconsistent pharmacokinetics would be expected, which may further hamper the therapeutic efficacy and generated some adverse effects. In addition, the extra N-methyl group of 3-(4,5-dibromo-2-methyl-3,6-dioxo-pyridazin-1-yl) propanoic acid (the moiety suitable for derivatizing to protein-drug conjugates) may suffer a metabolic problem of demethylation, which probably would result in poor PK and complicated metabolic profiles of ADCs. Due to the unsymmetrically structural natures of diBrPD, manufacture of the ADC products with this connector would be difficult to establish a high QC standard.

### SUMMARY OF THE DISCLOSURE

The present disclosure provides a novel class of symmetric bicyclic dibromopyridazinedione cysteine conjugated type linkers, among others, to avoid or minimize the aforementioned shortcomings.

In one aspect, the present disclosure provides a compound of formula (I):

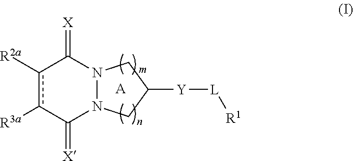

or a salt thereof, wherein:
Ring A is a 5 to 13-membered heterocycle;
m and n are each independently 1, 2, 3, 4, or 5;
[symbol] represents a double or single bond;
X and X′ are each independently O, S, or NR^{x}, wherein R^{x} is C1-C6 alkyl, C2-C6 alkenyl, or C2-C6 alkynyl;
Y is a bond, (CH₂)ᵢ (i=integer from 1 to 12), C(O), C(O)O, OC(O), C(O)NR^{a}, NR^{a}(CO), NR^{a}, O, S, S(O), S(O)₂, substituted or unsubstituted C6-C10 arylene, or substituted or unsubstituted 5 to 12 membered heteroarylene, or a combination thereof,
R^{2a} and R^{3a} are each independently hydrogen, halogen, —U—R², or —V—R³.
U and V are independently a bond, S, S(O), S(O)₂, O, NH, or CH₂;
L is a bond or linear or branched-chain linker comprising a group or moiety selected from substituted or unsubstituted alkylene, substituted or unsubstituted cyclylene, substituted or unsubstituted heterocyclylene, substituted or unsubstituted arylene, substituted or unsubstituted heteroarylene, O, S, NR^{a}, C(O), C(O)O, OC(O), C(O)NH, NHC(O), S(O), S(O)₂, (CH₂CH₂O)ⱼ, (OCH₂CH₂)ⱼ (PEGn, j=2 to 48), S—S, hydrazone, substituted or unsubstituted oligo peptide (e.g., Val-Cit, Gly-Gly-Phe-Gly, Val-Ala, Ala-Ala, Ala-Ala-Asn, Phe-Lys, Val-Lys, or Val-Arg), and combinations thereof, wherein R^{a} is hydrogen or C₁-C₆ alkyl, and wherein the linker L optionally comprises a self-immolative spacer;
R¹ is hydrogen, substituted or unsubstituted alkyl, substituted or unsubstituted cycloalkyl, substituted or unsubstituted aryl, substituted or unsubstituted heteroaryl, substituted or unsubstituted heterocyclyl, substituted or unsubstituted succinimidyl, or a functional moiety selected from: a leaving group (e.g., BSA, KLH, and OVA), a detectable moiety, an enzymatically active moiety, an affinity tag, a hapten, an immunogenic carrier (e.g., BSA, KLH, and OVA), radionuclides, photosensitizers, cytotoxins and their prodrugs, innate immune modulators, biopolymers, oligonucleotides, PROTAC degraders, antibiotics, and exotoxin;
R² and R³ are each independently selected from hydrogen, halogen, substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocyclyl, antibody, antigen, liposome, polymeric moiety, substituted or unsubstituted amino acid moiety, substituted or unsubstituted peptide moiety, DNA, RNA, virus or virus-like particles, and targeting ligand small molecule which carries a nucleophilic group or molecular moiety, such as —SH, —OH, —NH₂, guanidinyl, imidazolyl, indolyl, and carboxylic acid (—CO₂H);
or alternatively, R² and R³ together become R⁴, which together with U, V, and [symbol] forms a ring B as characterized in formula (II):

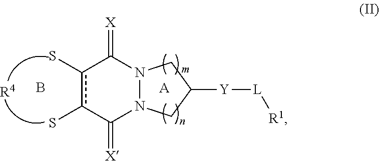

wherein R⁴ is selected from alkylene, alkenylene, alkynylene, arylene, heteroarylene, cycloalkylene, heterocyclylene, and combinations thereof, each optionally substituted; or a moiety of antibody, antigen, liposome, polymer, amino acid, peptide, DNA, RNA, virus, virus-like particles, or targeting ligand small molecule, wherein the targeting ligand small molecule optionally comprises a nucleophilic group selected from —SH, —OH, —NH₂, guanidinyl, imidazolyl, indolyl, and carboxylic acid, or a combination thereof.

In one aspect, the present disclosure provides a compound of Formula (II):

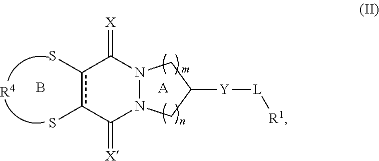

or a pharmaceutically acceptable salt thereof, wherein
Ring A is independently 5 to 13-membered carbocycles;
m and n are each independently 1, 2, 3, 4, or 5; Ring B comprises two nonnucleophilic groups (e.g., thiol groups derived from a disulfide bridge in a peptide, protein, or antibody;
X and X′ are each independently O, S, or NR^{x};
Y is a bond (CH₂)ᵢ (i=integer from 1 to 12), C(O), C(O)O, NR^{a}, O, S, S(O), S(O)₂, substituted or unsubstituted C6-C10 arylene, or substituted or unsubstituted 5 to 12 membered heteroarylene, or a combination thereof,
L is a bond or linear or branched-chain linker comprising a group or moiety selected from alkylene, O, S, NR^{a}, C(O), C(O)O, OC(O), C(O)NH, NHC(O), S(O), S(O)₂, (CH₂CH₂O)ⱼ, (OCH₂CH₂)ⱼ (PEGn, j=2 to 48), S—S, hydrazone, oligo peptide, (e.g., Val-Cit, Gly-Gly-Phe-Gly, Val-Ala, Ala-Ala, Ala-Ala-Asn, Phe-Lys, Val-Lys, or Val-Arg), and combinations thereof, wherein the linker optionally comprises a self-immolative spacer; R¹ is hydrogen, alkyl, cycloalkyl, aryl, succinimidyl, or a functional moiety selected from: a leaving group, a detectable moiety, an enzymatically active moiety, an affinity tag, a hapten, an immunogenic carrier, radionuclides, photosensitizers, cytotoxins and their prodrugs, innate immune modulators, biopolymers, oligonucleotides, PROTAC degraders, antibiotics, and exotoxin.
R^{x} is C1-C6 alkyl, C2-C6 alkenyl, or C2-C6 alkynyl;
R⁴ is selected from alkylene, alkenylene, alkynylene, arylene, heteroarylene, cycloalkylene, heterocyclylene, and combinations thereof, each optionally substituted; or a moiety of antibody, antigen, liposome, polymer, amino acid, peptide, DNA, RNA, virus, virus-like particles, or targeting ligand small molecule, wherein the targeting ligand small molecule optionally comprises a nucleophilic group selected from —SH, —OH, —NH₂, guanidine, imidazole, indole, carboxylic acid, or a combination thereof, and
R^{a} is hydrogen or C1-C6 alkyl.

In one aspect, the present disclosure provides a pharmaceutical composition comprising a compound according to any one of the embodiments disclosed herein and a pharmaceutically acceptable carrier.

In one aspect, the present disclosure provides a method of treating a disease or disorder, comprising administering to a subject in need thereof a compound according to any one of the embodiments disclosed herein, or a pharmaceutically acceptable salt or pharmaceutical composition thereof.

In one aspect, the present disclosure provides use of a compound according to any one of the embodiments disclosed herein, or a pharmaceutically acceptable salt thereof, in the manufacture of a medicament for treatment of a disease or disorder.

Other aspects or advantages of the present disclosure will be better appreciated by those skilled in the art in view of the following detailed description, drawings, examples, and claims.

### BRIEF DESCRIPTION OF THE DRAWINGS

FIG. 1 illustrates the regioisomeric issues of dibromopyridazindione (diBrPD) connector (FIG. 1a) and the solutions of current disclosure (FIG. 1b).

FIG. 2 shows a Hydrophobic Interaction Chromatograph (HIC) diagram of an antibody-SBC-CL075 (E13) conjugated that corresponds to drug-load species with DAR4.

FIG. 3 demonstrates a HIC diagram of an antibody-SBC-MMAF (E14) conjugated that corresponds to drug-load species with DAR4.

FIG. 4 depicts a HIC diagram of an antibody-diBrPD-CL075 conjugated that corresponds to drug-load species with DAR1, 2, 3, 4 and 5.

FIG. 5 shows a HIC diagram of an antibody-diBrPD-MMAF conjugated that corresponds to drug-load species with DAR3, 4, and 5.

### DETAILED DESCRIPTION OF THE DISCLOSURE

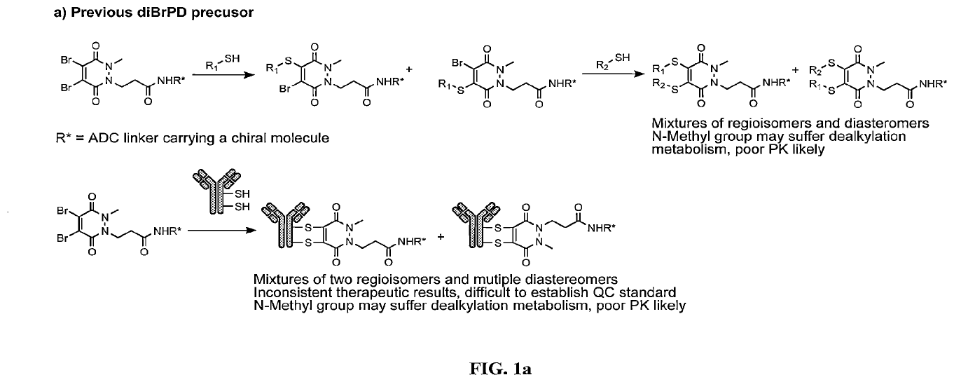

The pyridazinedione (PD) moiety has been shown to address many of the drawbacks associated with commonly employed Michael acceptors (e.g., maleimides) in the purpose of cysteine modification, providing a linker that is stable in serum for several days (i.e., PD-derived bioconjugates do not react with high concentrations of human serum albumin (HSA) and low concentrations of glutathione (GSH)). PD-protein constructs have also been shown to be cleavable in high concentrations of a reactive thiol (offering a proposed release mechanism under intracellular early endosomal conditions (i.e., a high GSH concentration between 1 and 10 mM, and a pH range 6.8-5.9). Furthermore, the PD scaffold can readily incorporate functional handles to link a spacer and payload for the preparation of antibody-drug conjugates (ADCs) (Peter A. Szijj et al., Drug Discovery Today: Technologies 2018, 30, 27; Calise Bahou et al., Org. Biomol. Chem., 2018, 16, 1359; Marcos Fernandez et al., Chem. Commun., 2020, 56, 1125; Calise Bahou et al., Org. Biomol. Chem., 2018, 16, 1359; Ofelia Feuillatre et al., ACS Omega 2020, 5, 1557-1565; WO2019034868). However, the existing pyridazinedione-based linkers suffer various aforementioned limitations, one of which is illustrated in (FIG. 1a).

While maintaining the advantages of pyridazinedione (PD), the present disclosure provides a new class of linkers to overcome the limitations of the existing pyridazinedione (PD)-based linkers, for example, by bearing a 2,3-dihydro-1H-pyrazolo[1,2-a]pyridazine-5,8-dione moiety in one case. These linkers can be synthesized from commercially available starting materials in 1-3 steps. The bromides on the dihydropyrazolopyridazinedione (6,7-dibromo-5,8-dioxo-2,3-dihydro-1H-pyrazolo[1,2-a]pyridazine) are highly reactive (16 h was reported for full conversion using diBrPD, which is longer than bromomaleimides) toward the thiols derived from the reduction of disulfide bridges in proteins (cysteine-specific modification). Compared to the monocyclic diBrPD linkers reported by UCL, this structurally symmetric bicyclic heterocyclic scaffold would solve the regioselective issues brought by the monocyclic system. We believe the Symmetric Bicyclic-dibromopyridazinedione Cysteine (“SBC”) linkers should have higher cysteine specificity and be pharmacokinetically superior (N-methyl group eliminated). In addition, the extra functional groups introduced by this type of connectors, such as carboxylic acid, amino group, and hydroxyl group, would provide opportunities of multi-functionalization of a single cysteine or disulfide bridging bioconjugation. This method has a broad substrate scope and allows the installation of a wide range of synthetic modifications on different protein scaffolds, including antibodies, without disturbing their native antigen-binding properties (FIG. 1b).

In one aspect, the present disclosure provides a compound of formula (I):

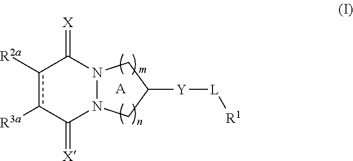

or a salt thereof, wherein:
Ring A is a 5 to 13-membered heterocycle;
m and n are each independently 1, 2, 3, 4, or 5;
[symbol] represents a double or single bond;
X and X′ are each independently O, S, or NR^{x};
Y is a bond, (CH₂)ᵢ (i=integer from 1 to 12), C(O), C(O)O, C(O)NR^{a}, NR^{a}, O, S, S(O), S(O)₂, substituted or unsubstituted C6-C10 arylene, or substituted or unsubstituted 5 to 12 membered heteroarylene, or a combination thereof,
R^{2a} and R^{3a} are each independently halogen, —U—R², or —V—R³.
U and V are independently a bond, S, O, NH, or CH₂;
L is a bond or linear or branched-chain linker comprising a group or moiety selected from alkylene, O, S, NR^{a}, C(O), C(O)O, OC(O), C(O)NH, NHC(O), S(O), S(O)₂, (CH₂CH₂O)ⱼ, (OCH₂CH₂)ⱼ (PEGn, j=2 to 48), S—S, hydrazone, oligo peptide, (e.g., Val-Cit, Gly-Gly-Phe-Gly, Val-Ala, Ala-Ala, Ala-Ala-Asn, Phe-Lys, Val-Lys, or Val-Arg), and combinations thereof, wherein the linker optionally comprises a self-immolative spacer; R¹ is hydrogen, alkyl, cycloalkyl, aryl, succinimidyl, or a functional moiety selected from: a leaving group, a detectable moiety, an enzymatically active moiety, an affinity tag, a hapten, an immunogenic carrier, radionuclides, photosensitizers, cytotoxins and their prodrugs, innate immune modulators, biopolymers, oligonucleotides, PROTAC degraders, antibiotics, and exotoxin;
R² and R³ are each independently selected from halogen, alkyl, alkenyl, alkynyl, aryl, heteroaryl, cycloalkyl, heterocyclyl, antibody, antigen, liposome, polymeric moiety, amino acid, peptide, DNA, RNA, virus or virus-like particles, targeting ligand small molecule which carries a nucleophilic group or molecular moiety, such as —SH, —OH, —NH₂, guanidine, imidazole, indole, and carboxylic acid;
or alternatively, R² and R³ together become R⁴ together with U, V, and [symbol] form a ring B as characterized in formula (II):

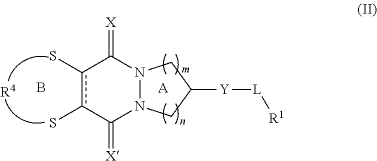

R^{x} is C1-C6 alkyl, C2-C6 alkenyl, or C2-C6 alkynyl;
R⁴ is selected from alkylene, alkenylene, alkynylene, arylene, heteroarylene, cycloalkylene, heterocyclylene, and combinations thereof, each optionally substituted; or a moiety of antibody, antigen, liposome, polymer, amino acid, peptide, DNA, RNA, virus, virus-like particles, or targeting ligand small molecule, wherein the targeting ligand small molecule optionally comprises a nucleophilic group selected from —SH, —OH, —NH₂, guanidine, imidazole, indole, carboxylic acid, or a combination thereof, and
R^{a} is hydrogen or C₁-C₆ alkyl.

In some embodiments, in the compound of formula (I) or (II), or a salt thereof:
Ring A is a 5 to 9-membered heterocycle; and
m and n are each independently 1, 2, or 3.

In some embodiments, in the compound of formula (I) or (II), or a salt thereof:
Ring A is a 5 to 7-membered heterocycle; and
m and n are each independently 1 or 2.

In some embodiments, in the compound of formula (I) or (II), or a salt thereof:
m=1, and n=1.

In some embodiments, in the compound of formula (I) or (II), or a salt thereof:
[symbol] is a double bond, and X and X′ are each O.

In some embodiments, in the compound of formula (I) or (II), or a salt thereof:
U and V are each a bond, and R² and R³ are each halogen.

In some embodiments, in the compound of formula (I) or (II), or a salt thereof:
R² and R³ are each bromo (Br).

In some embodiments, in the compound of formula (I) or (II), or a salt thereof:
U and V are each sulfur (S); and
R² and R³ are each independently alkyl, aryl, heteroaryl, cycloalkyl, heterocyclyl, or an amino acid moiety, each optionally substituted.

In some embodiments, in the compound of formula (I) or (II), or a salt thereof:
U and V are each sulfur (S); and
R² and R³ are each independently or together selected from antibody, antigen, liposome, polymeric moiety, amino acid, oligopeptide, DNA, RNA, virus or virus-like particles, and targeting ligand small molecule.

In some embodiments, in the compound of formula (I) or (II), or a salt thereof:
Y is C(O)O, C(O)NH, CH₂, O, S, NH;
L is a bond, alkylene, —(CH₂)ₖC(O)NH— (k is an integer selected from 1 to 8); and
R¹ is hydrogen, alkyl, cycloalkyl, aryl, or succinimidyl.

In some embodiments, in the compound of formula (I) or (II), or a salt thereof:
Y is C(O)NH;
L is a bond, —(CH₂)ₖC(O)NH— (k is an integer selected from 1 to 8), or oligopeptide moiety, or a combination thereof; and
R¹ is selected from hydrogen, alkyl, cycloalkyl, aryl, a detectable moiety, an enzymatically active moiety, an affinity tag, a hapten, an immunogenic carrier, radionuclides, photosensitizers, cytotoxins and their prodrugs, innate immune modulators, biopolymers, oligonucleotides, PROTAC degraders, antibiotics, and exotoxin.

In some embodiments, in the compound of formula (I) or (II), or a salt thereof, the oligopeptide moiety is selected from Val-Cit, Gly-Gly-Phe-Gly, Val-Ala, Ala-Ala, Ala-Ala-Asn, Phe-Lys, Val-Lys, and Val-Arg.

In some embodiments, in the compound of formula (I) or (II), or a salt thereof, the self-immolative spacer comprises a para-aminobenzyloxycarbonyl (PABC) moiety or a PABC-type moiety that can lead to electron cascade-mediated self-immolation, or a cyclization-mediated self-immolation.

In one aspect, the present disclosure provides a compound of Formula (II):

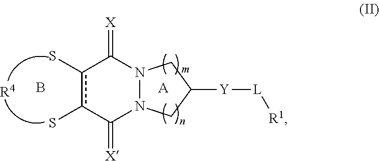

or a pharmaceutically acceptable salt thereof, wherein
Ring A is independently 5 to 13-membered carbocycles;
m and n are each independently 1, 2, 3, 4, or 5; Ring B comprises two nonnucleophilic groups (e.g., thiol groups derived from a disulfide bridge in a peptide, protein, or antibody;
X and X′ are each independently O, S, or NR^{x};
Y is a bond (CH₂)ᵢ (i=integer from 1 to 12), C(O), C(O)O, NR^{a}, O, S, S(O), S(O)₂, substituted or unsubstituted C6-C10 arylene, or substituted or unsubstituted 5 to 12 membered heteroarylene, or a combination thereof,
L is a bond or linear or branched-chain linker comprising a group or moiety selected from alkylene, O, S, NR a, C(O), C(O)O, OC(O), C(O)NH, NHC(O), S(O), S(O)₂, (CH₂CH₂O)ⱼ, (OCH₂CH₂)ⱼ (PEGn, j=2 to 48), S—S, hydrazone, oligo peptide, (e.g., Val-Cit, Gly-Gly-Phe-Gly, Val-Ala, Ala-Ala, Ala-Ala-Asn, Phe-Lys, Val-Lys, or Val-Arg), and combinations thereof, wherein the linker optionally comprises a self-immolative spacer; R¹ is hydrogen, alkyl, cycloalkyl, aryl, succinimidyl, or a functional moiety selected from: a leaving group, a detectable moiety, an enzymatically active moiety, an affinity tag, a hapten, an immunogenic carrier, radionuclides, photosensitizers, cytotoxins and their prodrugs, innate immune modulators, biopolymers, oligonucleotides, PROTAC degraders, antibiotics, and exotoxin.

R^{x} is C1-C6 alkyl, C2-C6 alkenyl, or C2-C6 alkynyl;
R⁴ is selected from alkylene, alkenylene, alkynylene, arylene, heteroarylene, cycloalkylene, heterocyclylene, and combinations thereof, each optionally substituted; or a moiety of antibody, antigen, liposome, polymer, amino acid, peptide, DNA, RNA, virus, virus-like particles, or targeting ligand small molecule, wherein the targeting ligand small molecule optionally comprises a nucleophilic group selected from —SH, —OH, —NH₂, guanidine, imidazole, indole, carboxylic acid, or a combination thereof, and
R^{a} is hydrogen or C1-C6 alkyl.

In some embodiments, in the compound of formula (II), or a salt thereof:
Ring A is a 5 to 9-membered heterocycle; and
m and n are each independently 1, 2, or 3.

In some embodiments, in the compound of formula (II), or a salt thereof:
Ring A is a 5 to 7-membered heterocycle; and
m and n are each independently 1 or 2.

In some embodiments, in the compound of formula (II), or a salt thereof:
m=1, and n=1.

In some embodiments, in the compound of formula (II), or a salt thereof:
[symbol] is a double bond, and X and X′ are each O.

In some embodiments, in the compound of formula (II), or a salt thereof:
U and V are each a bond, and R² and R³ are each halogen.

In some embodiments, in the compound of formula (II), or a salt thereof:
R² and R³ are each bromo (Br).

In some embodiments, in the compound of formula (II), or a salt thereof:
U and V are each sulfur (S); and
R² and R³ are each independently alkyl, aryl, heteroaryl, cycloalkyl, heterocyclyl, or an amino acid moiety, each optionally substituted.

In some embodiments, in the compound of formula (II), or a salt thereof:
U and V are each sulfur (S); and
R² and R³ are each independently or together selected from antibody, antigen, liposome, polymeric moiety, amino acid, oligopeptide, DNA, RNA, virus or virus-like particles, and targeting ligand small molecule.

In some embodiments, in the compound of formula (II), or a salt thereof:
Y is C(O)O, C(O)NH, CH₂, O, S, or NH;
L is a bond, alkylene, or —(CH₂)ₖC(O)NH—, wherein k is an integer selected from 1 to 8; and
R¹ is hydrogen, alkyl, cycloalkyl, aryl, or succinimidyl.

In some embodiments, in the compound of formula (II), or a salt thereof:
Y is C(O)NH;
L is a bond, —(CH₂)ₖC(O)NH—, wherein k is an integer selected from 1 to 8, or oligopeptide moiety, or a combination thereof, and
R¹ is selected from hydrogen, alkyl, cycloalkyl, aryl, a detectable moiety, an enzymatically active moiety, an affinity tag, a hapten, an immunogenic carrier, radionuclides, photosensitizers, cytotoxins and their prodrugs, innate immune modulators, biopolymers, oligonucleotides, PROTAC degraders, antibiotics, and exotoxin.

In some embodiments, in the compound of formula (II), or a salt thereof, the oligopeptide moiety is selected from Val-Cit, Gly-Gly-Phe-Gly, Val-Ala, Ala-Ala, Ala-Ala-Asn, Phe-Lys, Val-Lys, and Val-Arg.

In some embodiments, in the compound of formula (II), or a salt thereof, the self-immolative spacer comprises a para-aminobenzyloxycarbonyl (PABC) moiety or a PABC-type moiety (e.g., ortho-aminobenzyl, ortho-hydroxybenzyl, and para-hydroxybenzyl) that can lead to electron cascade-mediated self-immolation, or a cyclization-mediated self-immolation.

In one aspect, the present disclosure provides a pharmaceutical composition comprising a compound according to any one of the embodiments disclosed herein and a pharmaceutically acceptable carrier.

In one aspect, the present disclosure provides a method of treating a disease or disorder, comprising administering to a subject in need thereof a compound according to any one of the embodiments disclosed herein, or a pharmaceutically acceptable salt or pharmaceutical composition thereof.

In one aspect, the present disclosure provides use of a compound according to any one of the embodiments disclosed herein, or a pharmaceutically acceptable salt thereof, in the manufacture of a medicament for treatment of a disease or disorder.

In some embodiments, illustrative examples of the compounds of the invention include, but are not limited to compounds E01-E24 listed below:

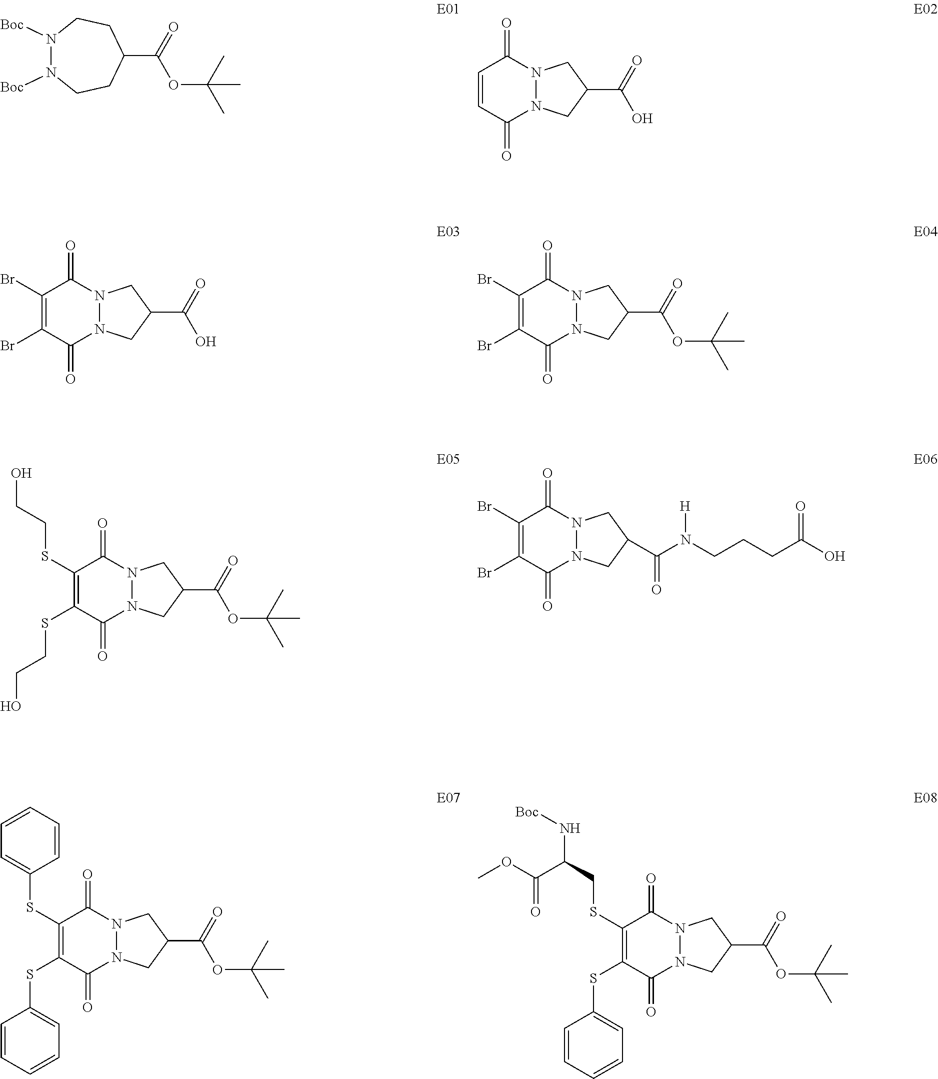

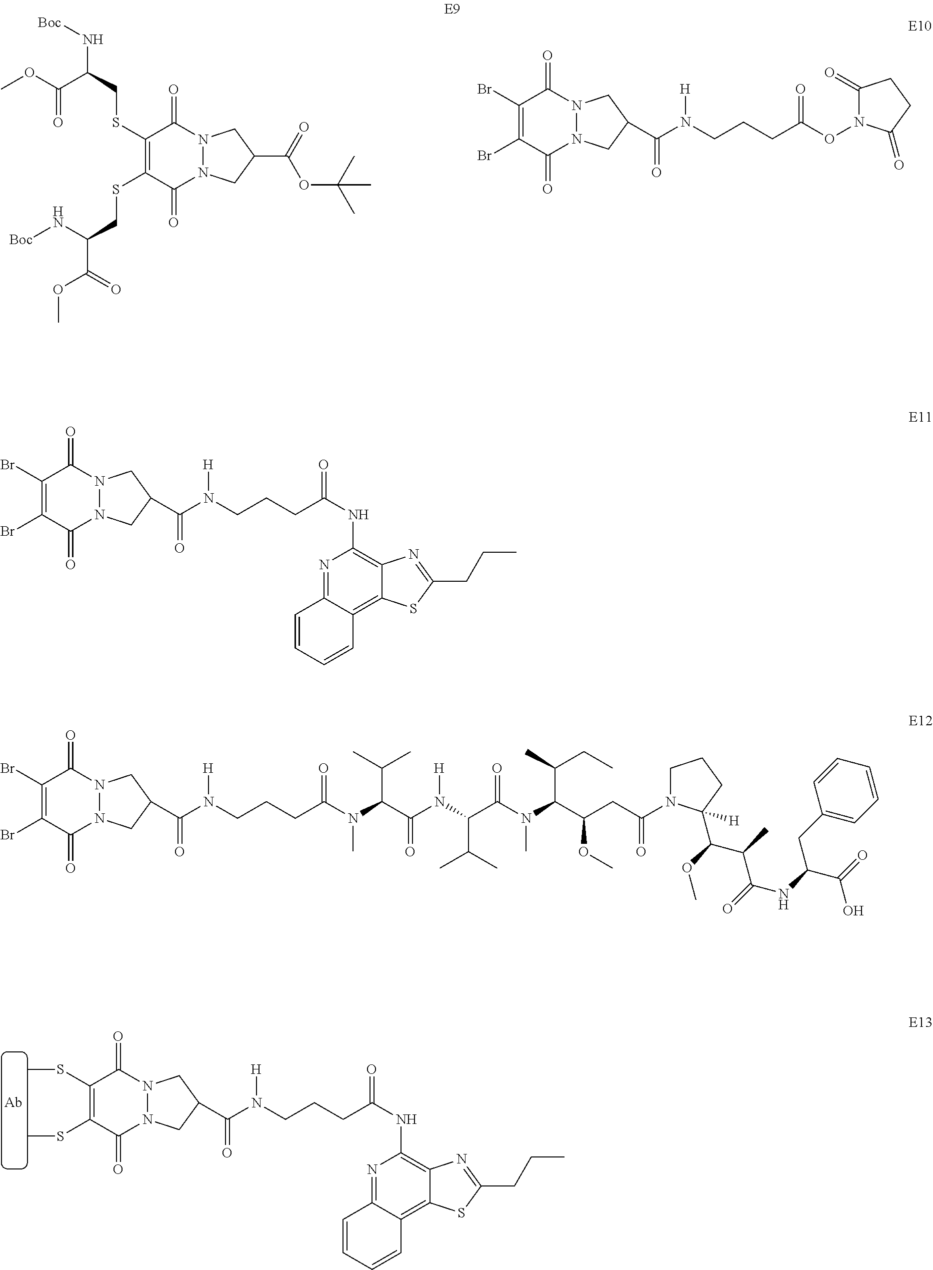 [SMILES: COC(=O)[C@H](CSc1c(SC[C@H](NC(=O)OC(C)(C)C)C(=O)OC)c(=O)n2n(c1=O)CC(C(=O)OC(C)(C)C)C2)NC(=O)OC(C)(C)C ; O=C(CCCNC(=O)C1Cn2c(=O)c(Br)c(Br)c(=O)n2C1)ON1C(=O)CCC1=O ; CCCc1nc2c(NC(=O)CCCNC(=O)C3Cn4c(=O)c(Br)c(Br)c(=O)n4C3)nc3ccccc3c2s1 ; CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCNC(=O)C1Cn2c(=O)c(Br)c(Br)c(=O)n2C1)C(C)C ; CCCc1nc2c(NC(=O)CCCNC(=O)C3Cn4c(=O)c(SC)c(SC)c(=O)n4C3)nc3ccccc3c2s1]

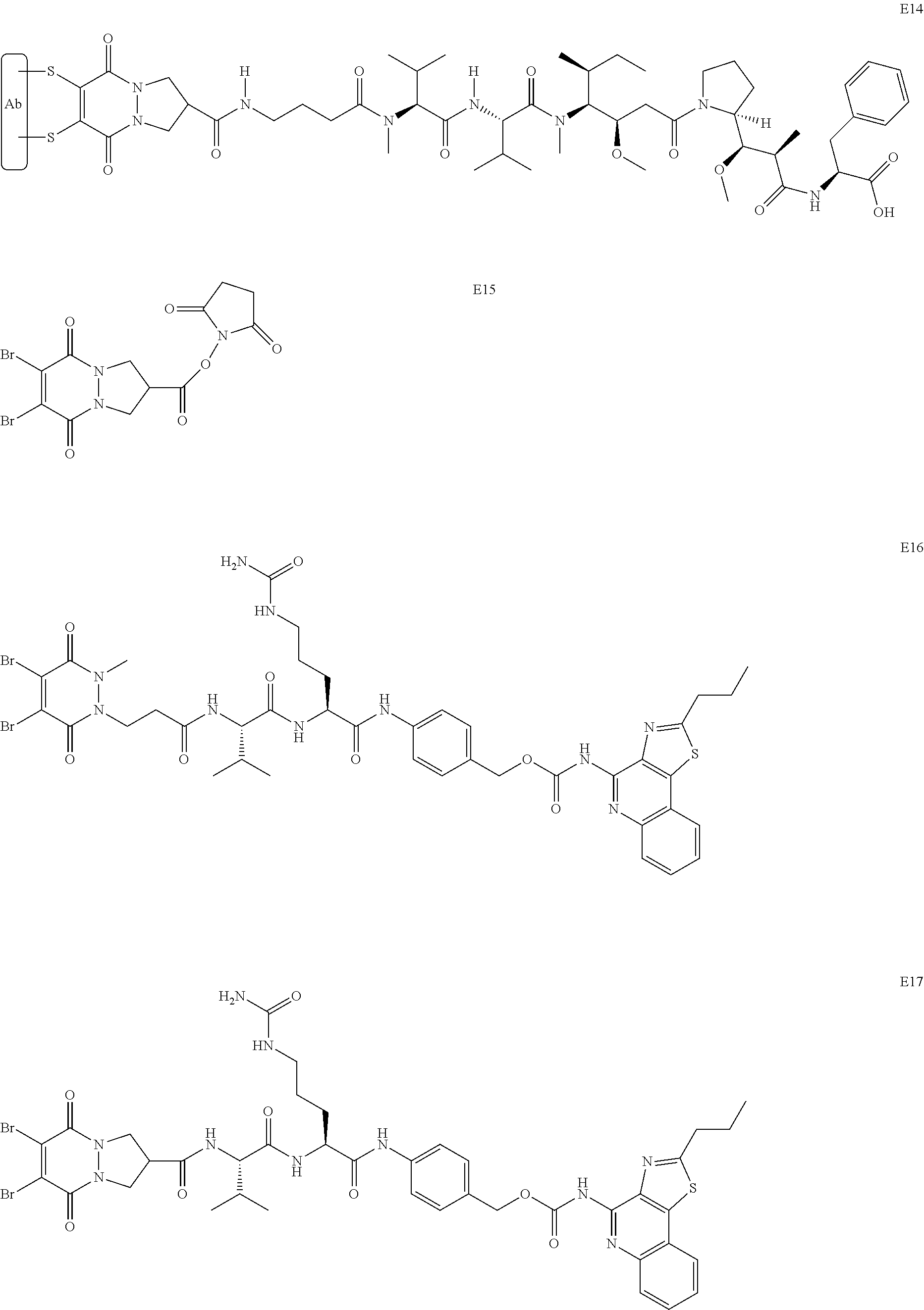 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCNC(=O)C1Cn2c(=O)c(SC)c(SC)c(=O)n2C1)C(C)C ; O=C(ON1C(=O)CCC1=O)C1Cn2c(=O)c(Br)c(Br)c(=O)n2C1 ; CCCc1nc2c(NC(=O)OCc3ccc(NC(=O)[C@H](CCCNC(N)=O)NC(=O)[C@@H](NC(=O)CCn4c(=O)c(Br)c(Br)c(=O)n4C)C(C)C)cc3)nc3ccccc3c2s1 ; CCCc1nc2c(NC(=O)OCc3ccc(NC(=O)[C@H](CCCNC(N)=O)NC(=O)[C@@H](NC(=O)C4Cn5c(=O)c(Br)c(Br)c(=O)n5C4)C(C)C)cc3)nc3ccccc3c2s1]

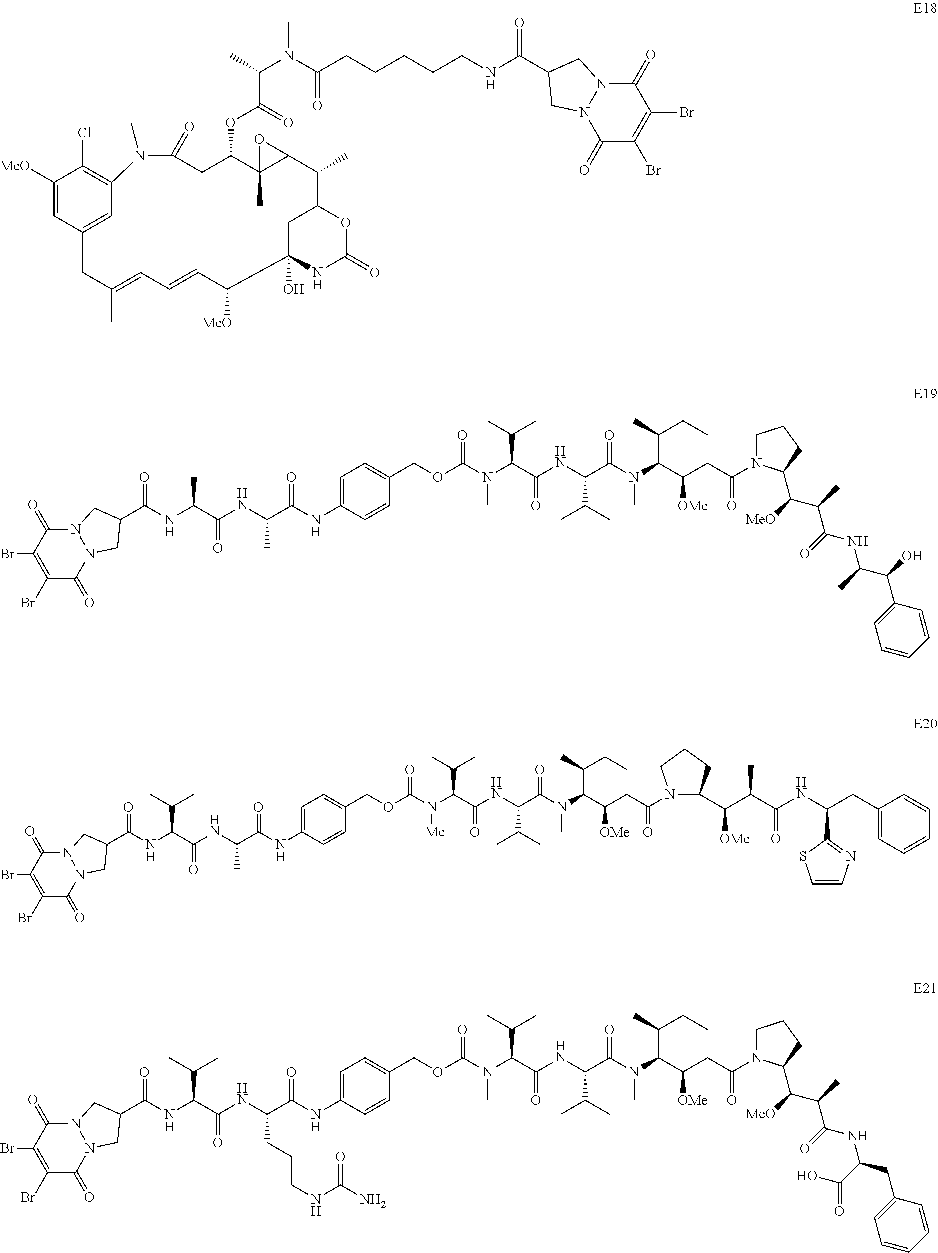

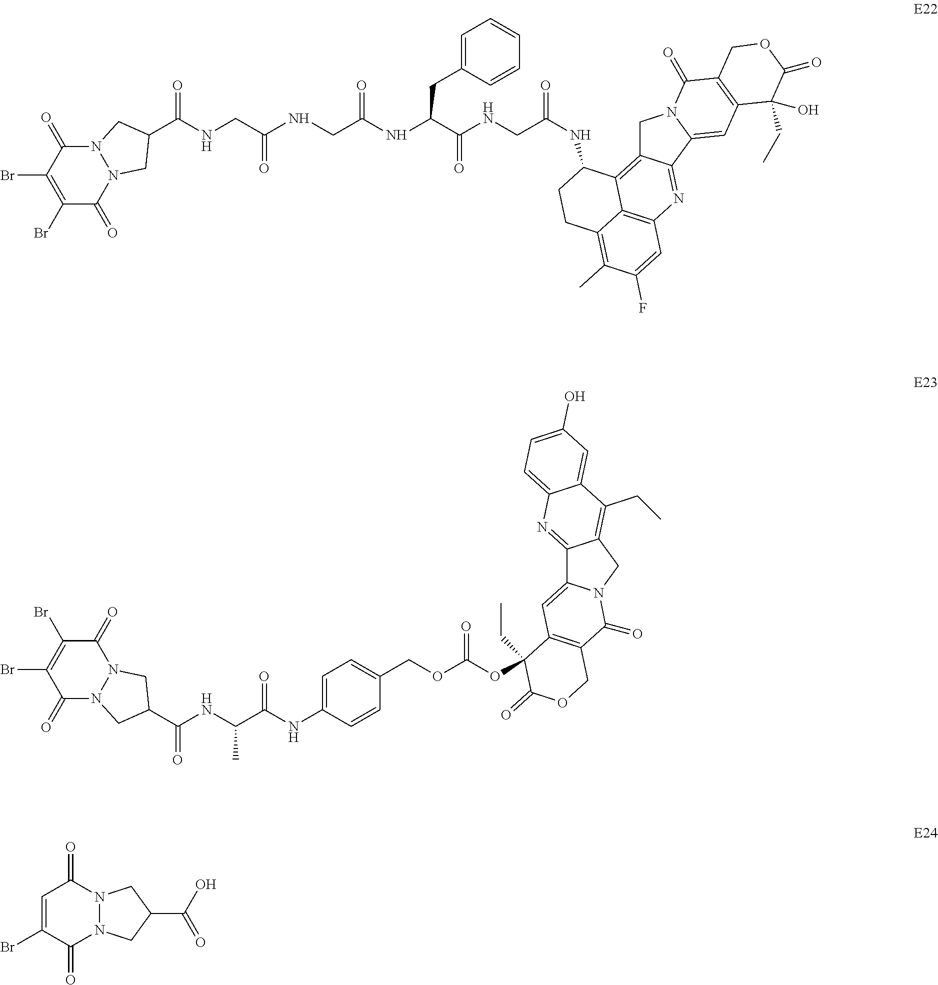 [SMILES: CC[C@@]1(O)C(=O)OCc2c1cc1n(c2=O)Cc2c-1nc1cc(F)c(C)c3c1c2[C@@H](NC(=O)CNC(=O)[C@H](Cc1ccccc1)NC(=O)CNC(=O)CNC(=O)C1Cn2c(=O)c(Br)c(Br)c(=O)n2C1)CC3 ; CCc1c2c(nc3ccc(O)cc13)-c1cc3c(c(=O)n1C2)COC(=O)[C@@]3(CC)OC(=O)OCc1ccc(NC(=O)[C@H](C)NC(=O)C2Cn3c(=O)c(Br)c(Br)c(=O)n3C2)cc1 ; O=C(O)C1Cn2c(=O)cc(Br)c(=O)n2C1]

In some embodiments, this disclosure provides a compound selected from compounds E1 through E24, or a salt or stereoisomer thereof, or a pharmaceutical composition comprising any of the compounds selected from compounds E1 through E24.

### Definitions

As used herein, the term “moiety,” “chemical moiety,” “molecular moiety,” or the like, means a characteristic (often major) portion of another molecule as an integral part of the subject structure being defined, where the moiety is covalently bonded to the rest of the structure through one, two, or three positions on the molecule after removal of one, two, or three peripheral atoms or groups from such positions of the molecule. For example, a peptide contains multiple amino acid moieties. An amino acid moiety inside the peptide chain or chains (if branched) may be covalently bonded with two or three other amino acid moieties, whereas an amino acid moiety at an end of the peptide is covalently bonded only with its adjacent amino acid moiety.

As used herein, the term “detectable moiety” means a moiety which is capable of generating detectable signals and are also commonly known in the art as “tags”, “probes” and “labels”. Examples of detectable moieties include chromogenic moieties, fluorescent moieties, radioactive moieties and electrochemically active moieties.

A chromogenic moiety is a moiety which is coloured or becomes coloured when it is incorporated into a conjugate and the conjugate subsequently interacts with a secondary target species. Examples include porphyrins, polyenes, polyynes and polyaryls.

A fluorescent moiety is a moiety which comprises a fluorophore. Examples of fluorescent compounds include Alexa Fluor dyes, cyanine and merocyanine, boron-dipyrromethene dyes, ATIO dyes, fluorescein and its derivatives (rhodamine, coumarin, sulforhodamine 101 acid chloride (Texas Red) and dansyl), rhodamine and its derivatives, naphthalene derivatives, pyridyloxazole, nitrobenzoxadiazole and benzoxadiazole derivatives, coumarin and its derivatives, pyrene derivatives, and Oregon green, eosin, Cascade blue and Nile red.

A radioactive moiety refers to “radionuclide”, “radioactive nuclide”, “radioisotope”, “radioactive isotope”, “radioactive compound” or “radiolabel” and is a moiety that comprises a radionuclide and is an atom that excess nuclear energy. Radionuclides can be used for their radiation (e.g. irradiation to damage or kill pathogenic cells) or for the combination of chemical properties and their radiation (e.g. tracers and biopharmaceuticals). Some non-limiting examples of radioisotopes include gallium-68, Copper-64, lutetium-177, iodine-131, iodine-125, bismuth-212, yttrium-90, yttrium-88, technetium-99m, copper-67, rhenium-188, rhenium-186, gallium-66, gallium-67, indium-111, indium-114m, indium-114, boron-10, tritium (hydrogen-3), carbon-14, sulfur-35, fluorine-18 and carbon-11. Fluorine-18 and carbon-11, for example, are frequently used in positron emission tomography.

An electrochemically active moiety is a moiety that is capable of generating an electrochemical signal under an ampere metric or volta metric method and is capable of existing in at least two distinct redox states. Examples of electrochemically active moiety include dopamine hydrochloride, ascorbic acid, phenol and derivatives, benzoquinones and derivatives.

As used herein, the term “affinity tag” means a chemical moiety which is capable of interacting with an “affinity partner”, a second chemical moiety presented in a single sample, for example, between an enzyme and its substrate. Example of affinity tag/affinity partner pair that is particularly widely used in biochemistry are amylase/maltose binding protein, glutathione/glutathione-S-transferase and metal (biotin/streptavidin e.g., nickel or cobalt)/poly (His).

As used herein, the term, the term “hapten” means a moiety which is a low molecular weight non-protein agent and comprises an epitope and becomes an immune stimulator when linked to an immunogenic carrier molecule.

As used herein, the term “immunogenic carrier” means an antigen that is able to facilitate an immune response. Examples of immunogenic carriers include proteins, liposomes, synthetic or natural polymeric moieties (such as dextran, agarose, polylysine and polyglutamic acid moieties) and synthetically designed organic moieties. Commonly used protein immunogenic carriers include keyhole limpet hemocyanin, bovine serum albumin, aminoethylated or cationised bovine serum albumin, thyroglobulin, ovalbumin and various toxoid proteins such as tetanus toxoid and diphtheria toxoid. Synthetically designed organic molecule carriers include the multiple antigentic peptide (MAP).

As used herein, the term “photosensitizers” means a moiety that is capable of absorbing light and transferring the energy from the incident light into another nearby molecule. A vast number of photosensitizers have been used as Photoimmunotherapy, such as porphyrins, chlorins and phthalocyanine dyes.

As used herein, the term “cytotoxins” means a moiety that is capable of being cytotoxic to cells by disrupting tubulin, damaging DNA, inhibiting topoisomerases, and preventing other essential cell processes. Exemplary cytotoxins and their prodrugs include maytansinoids, auristatins, dolastatins, tubulysins, eribulin, cryptomycins, topoisomerase inhibitors, durcarmycins, nemorubicin, pyrrolobenzodiazepine (PBD)s, calicheamicins, camptothecins, amatoxins, antimitotic EG5 Inhibitors, apoptosis inducers, thailanstatins, inhibitors of the nicotinamide phosphoribosyltransferase, carmaphycin.

As used herein, the term “innate immune modulators” means a moiety of pathogen-associated molecular patterns (PAMPs) or danger-associated molecular patterns (DAMPs) that binds to pattern recognition receptors (PRRs). The recognition of PAMPs or DAMPs by the PRRs triggers an inflammatory response that include the secretion of cytokines/chemokines, the induction of antimicrobial peptides, pyroptotic cell death and the recruitment of phagocytic cells. Exemplary innate immune modulators include tumor necrosis factor (TNF) superfamily ligands, C-type lectin receptors (CLRs) ligands, retinoic acid-inducible gene I (RIG-1)-like receptors (RLRs) ligands, stimulator of interferon gene (STING) ligands, toll-like receptors (TLRs) ligands, cytosolic DNA sensors (CDS) ligands.

Innate immune modulators suitable for use with the compositions and methods described herein include, without limitation, CU-T12-9, Pam3CSK4, FSL-1 (Pam2CGDPKHPKSF), poly (I:C), LPS (Lipopolysaccharide), MPLA (monophosphoryl Lipid A), CRX-527, FLA (flagellin), CL075 (also named 3M002, a thiazoloquinolone derivative), CL097, CL264, CL307, CL429, Gardiquimod, R837 (imiquimod), R0848 (resiquimod), Loxoribine, TL8-506, CU-CPT9a, ODN2088 (CpG oligodeoxynucleotides 2088), ODN4084, ODN INH-18, ODN1585, ODN2216, ODN2336, ODN1668, ODN2006, ODN1826, ODN BW006, ODN D-SL01, ODN2395, ODN M362, ODN SL03, C12-iE-DAP, C14-Tri-LAN-Gly, iE-DAP, iE-Lys, Tri-DAP, MDP (Muramyl dipeptide), L18-MDP, Beta-glucan, Curdlan, HKCA (heat-killed preparation of C. albicans), laminarin, pustulan, scleroglucan, Zymosan, furfurman, GlcC14C18, Beta-glucosylceramide, TDB (Trehalose-6,6-dibehenate), 2′3′-cGAMP, 3′3′-cGAMP, c-di-AMP, 2′2′-cGAMP. DMXAA, dsDNA, G3-YSD (unpaired guanosine trimers-ended Y-form Short DNA), HSV-60, ISD (interferon stimulatory DNA).

As used herein, the term of “biopolymers” means a moiety of natural polymers produced by the cells of living organisms. Biopolymers consist of monomeric units that are covalently bonded to form larger molecules. Exemplary biopolymer includes high molecular weight phosphorylcholine polymer.

As used herein, the term “enzymatically active moiety” means an enzyme, a substrate for an enzyme or a cofactor for an enzyme that is capable of acting as biological catalysts or/and therapeutic agent to modify microenvironmental condition throughout accelerating chemical reactions. Exemplary of microenvironment modifiers and therapeutic enzymes is urease, a member of superfamily of amidohydrolases and phosphotriesterases.

As used herein, the term of “oligonucleotides” means a short DNA or RNA molecules, oligomers that capable of being a therapeutic agent, exemplary therapeutic oligonucleotides include Myotonic dystrophy type 1 antisense oligonucleotides that degrade DMPK transcripts and reduce levels of DMPK mRNA in a durable.

“PROTAC degraders” form an enzymatic complex in the cell that acts in a catalytic fashion to knockdown the target protein. Typically comprising binding moieties for an E3 ubiquitin ligase and a target protein joined by a linker. Exemplary protein degraders include bromodomain-containing protein 4 (BRD4) degrader.

As used herein, the term of “antibiotics” means an agent that is chemical substance produced by a living microorganism, that is detrimental to other microorganisms. Antibiotics produce their effects by inhibiting bacterial cell wall synthesis or function; or inhibiting protein synthesis in bacteria.

As used herein, the term “exotoxin” means a moiety that is secreted by bacteria, Well-known exotoxins include botulinum toxin and Corynebacterium diphtheriae toxin,

The term “antibody” as used herein refers to a glycoprotein comprising at least two heavy (H) chains and two light (L) chains inter-connected by disulfide bonds. Each heavy chain is comprised of a heavy chain variable region (VH) and a heavy chain constant region. The heavy chain constant region is comprised of three domains, CH₁, CH₂ and CH₃. Each light chain is comprised of a light chain variable region (VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system and the first component (C1q) of the classical complement system. The term “antibody” includes for example, monoclonal antibodies, human antibodies, humanized antibodies, camelized antibodies, chimeric antibodies, single-chain Fvs (scFv), disulfide-linked Fvs (sdFv), Fab fragments, F (ab′) fragments, and anti-idiotypic (anti-Id) antibodies, and epitope-binding fragments of any of the above. The antibodies can be of any isotype (e.g., IgG, IgE, IgM, IgD, IgA and IgY), class (e.g., IgG1, IgG2, IgG3, IgG4, IgA1 and IgA2) or subclass.

The phrase “antibody fragment”, as used herein, refers to one or more portions of an antibody that retain the ability to specifically interact with an epitope. Examples of binding fragments include, but are not limited to, a Fab fragment, a monovalent fragment consisting of the VL, VH, CL and CHI domains; a F(ab)2 fragment, a bivalent fragment comprising two Fab fragments linked by a disulfide bridge at the hinge region; a Fd fragment consisting of the VH and CHI domains; a Fv fragment consisting of the VL and VH domains of a single arm of an antibody; a dAb fragment (Ward et al., (1989) Nature 341:544-546), which consists of a VH domain; and an isolated complementarity determining region (CDR).

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies.

Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH—CH 1-VH—CH1) which, together with complementary light chain polypeptides, form a pair of antigen binding regions (Zapata et al., (1995) Protein Eng. 8:1057-1062; and U.S. Pat. No. 5,641,870).

The term “antigen” as used herein refers to a site on a polypeptide macromolecule to which an antibody binds, forming an antibody-antigen complex. The proteins useful as antigens herein can be any native form the proteins from any vertebrate source, including mammals such as primates (e.g. humans) and rodents (e.g. mice and rats), unless otherwise indicated.

The term “epitope” denotes the site on an antigen, either proteinaceous or non-proteinaceous, to which an antibody binds. Epitopes can be formed from contiguous amino acid stretches (linear epitope) or comprise non-contiguous amino acids (conformational epitope), e.g., coming in spatial proximity due to the folding of the antigen, i.e., by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents.

As used herein, the term “ligand” means a moiety that is able to interact with a biomolecule (for example, a protein) in such a way as to modify the functional properties of the biomolecule. Typically, the ligand is a moiety that binds to a site on a target protein. The interaction between the ligand and the biomolecule is typically non-covalent. For example, the interaction may be through ionic bonding, hydrogen bonding or van der Waals' interactions. However, it is also possible for some ligands to form covalent bonds to biomolecules. Typically, a ligand is capable of altering the chemical conformation of the biomolecule when it interacts with it.

As used herein, the term “liposome” means a structure composed of phospholipid bilayers which have amphiphilic properties. Liposomes suitable for use in accordance with the present invention include unilamellar vesicles and multilamellar vesicles.

As used herein, the term “polymeric moiety” means a single polymeric chain (branched or unbranched), which is derived from a corresponding single polymeric molecule. Polymeric moieties may be natural polymers or synthetic polymers. Typically, though, the polymeric molecules are not polynucleotides.

As is well known in the biochemical field, creation of conjugates comprising a polymeric moiety is useful in many in vivo and in vitro applications. For example, various properties of a macromolecule such as a protein can be modified by attaching a polymeric moiety thereto, including solubility properties, surface characteristics and stability in solution or on freezing. Another common application involves conjugating a polymeric moiety to a biologically active compound such as a drug with the aim of enhancing biocompatibility, reducing, or eliminating immune response on administration, and/or increasing in vivo stability.

A person of skill in the art would therefore recognize that the methodology of the present invention can be used to prepare a conjugate comprising a polymeric moiety, which conjugate can then be used in any known application for polymeric-moiety-containing conjugates. A person of skill in the art would easily be able to select suitable polymeric moieties for use in accordance with the present invention, on the basis of those polymeric moieties used routinely in the art.

The nature of the polymeric moiety will therefore depend upon the intended use of the conjugate molecule. Exemplary polymeric moieties for use in accordance with the present invention include polysaccharides, polyethers, polyamino acids (such as polylysine), polyvinyl alcohols, polyvinylpyrrolidinones, poly(meth)acrylic acid and derivatives thereof, polyurethanes and polyphosphazenes. Typically, such polymers contain at least ten monomeric units. Thus, for example, a polysaccharide typically comprises at least ten monosaccharide units.

Two particularly preferred polymeric molecules are dextran and polyethylene glycol (“PEG”), as well as derivatives of these molecules (such as monomethoxypolyethylene glycol, “mPEG”). Preferably, the PEG or derivative thereof has a molecular weight of less than 20,000. Preferably, the dextran or derivative thereof has a molecular weight of 10,000 to 500,000. In one preferred embodiment, the compounds of the present invention comprise a biologically active moiety, for example a drug, and a PEG or derivative thereof.

As used herein, the term “amino acid” means a molecule containing both an amine functional group and a carboxyl functional group. However, preferably the amino acid is an α-amino acid. Preferably, the amino acid is a proteinogenic amino acid, i.e., an amino acid selected from alanine, arginine, asparagine, aspartic acid, cysteine, glutamic acid, glutamine, glycine, histidine, isoleucine, leucine, lysine, methionine, proline, phenylalanine, pyrrolysine, selenocysteine, serine, threonine, tryptophan, tyrosine, and valine. However, the amino acid can also be a non-proteinogenic amino acid. Examples of non-proteinogenic amino acids include lanthionine, 2-aminoisobutyric acid, dehydroalanine, gamma-aminobutyric acid, ornithine, citrulline, canavanine and mimosine. A particularly preferred amino acid according to the present invention is cysteine.

As used herein, the term “peptide” means a polymeric moiety made up of amino acid residues. As a person of skill in the art will be aware, the term “peptide” is typically used in the art to denote a polymer of relatively short length and the term “protein” is typically used in the art to denote a polymer of relatively long length. As used herein, the convention is that a peptide comprises up to 50 amino acid residues whereas a protein comprises more than 50 amino acids. However, it will be appreciated that this distinction is not critical since the functional moieties identified in the present application can typically represent either a peptide or a protein.

As used herein, the term “polypeptide” is used interchangeable with “protein”.

As used herein, a peptide or a protein can comprise any natural or non-natural amino acids. For example, a peptide or a protein may contain only a-amino acid residues, for example corresponding to natural a-amino acids. Alternatively, the peptide or protein may additionally comprise one or more chemical modifications. For example, the chemical modification may correspond to a post-translation modification, which is a modification that occurs to a protein in vivo following its translation, such as an acylation (for example, an acetylation), an alkylation (for example, a methylation), an amidation, a biotinylation, a formylation, glycosylation, a glycation, a hydroxylation, an iodination, an oxidation, a sulfation or a phosphorylation. A person of skill in the art would of course recognize that such post-translationally modified peptides or proteins still constitute a “peptide” or a “protein” within the meaning of the present invention. For example, it is well established in the art that a glycoprotein (a protein that carries one or more oligosaccharide side chains) is a type of protein.

As used herein, the term “DNA” means a deoxyribonucleic acid made up of one or more nucleotides. The DNA may be single stranded or double stranded. Preferably, the DNA comprises more than one nucleotide.

As used herein, the term “RNA” means a ribonucleic acid comprising one or more nucleotides. Preferably, the RNA comprises more than one nucleotide.

As used herein, the term “Virus-like particles (VLPs)” means multiprotein structure that mimics the organization and conformation of authentic native viruses but lack the viral genome. VLPs are useful as vaccines. VLPs contain repetitive, high density displays of viral surface proteins that present conformational viral epitopes that can elicit strong T cell and B cell immune responses; the particles' small radius of roughly 20-200 nm allows for sufficient draining into lymph nodes. Since VLPs cannot replicate, they provide a safer alternative to attenuated viruses. VLPs were used to develop FDA-approved vaccines for Hepatitis B and human papillomavirus, which are commercially available.

As used herein, the term “targeting ligand small molecule” means a low molecular weight (<900 daltons) organic compound that may regulate a biological process, with a size on the order of 1 nm. An example of targeting ligand small molecule is a HSP90 binding small molecule.

A “linker” or “linker group” is a group which is capable of covalently linking one chemical moiety (e.g., an antibody) to another (e.g., a functional moiety). Two main categories of clinkers have been described, non-cleavable linkers and cleavable linkers such as disulfide-containing, hydrazone, enzymatic cleavage linkers with self-immolation spacer. Examples of linker groups appropriate for use in accordance with the present invention are common general knowledge in the art and described in standard reference textbooks such as “Bioconjugate Techniques” (Greg T. Hermanson, Academic Press Inc., 1996), the content of which is herein incorporated by reference in its entirety.

An “antibody-drug conjugate”, or “ADC” is an antibody that is conjugated to one or more (typically 1 to 4) cytotoxins, each through a linker. The antibody is typically a monoclonal antibody specific to a cancer antigen.

The antibody conjugation reactive terminus of the linker is typically a site that is capable of conjugation to the antibody through a cysteine thiol or lysine amine group on the antibody, and so is typically a thiol-reactive group such as a double bond (as in maleimide) or a leaving group such as a chloro, bromo, or iodo, or an R-sulfanyl group, or an amine-reactive group such as a carboxyl group.

The term “alkyl” in the present invention refers to a linear or branched saturated hydrocarbon (i.e., free of double bonds or triple bonds). Alkyl group can have 1 to 9, sometimes preferably 1 to 6, and sometimes more preferably 1 to 4, carbon atoms (when appearing in the present invention, the numerical range of “1 to 9” refers to any integer in this range, for example, “1 to 9 carbon atoms” means that the alkyl group can contain 1 carbon atom, 2 carbon atoms, 3 carbon atoms, . . . , up to 9 carbon atoms. At the same time, the definition of alkyl also includes alkyl groups with no specified chain length). The alkyl group can be a medium-sized alkyl group containing 1 to 9 carbon atoms. Representative examples of alkyl group include, but are not limited to, methyl, ethyl, n-propyl, isopropyl, n-butyl, isobutyl, sec-butyl, tert-butyl, n-pentyl, n-hexyl, n-heptyl, 2-methylhexyl, n-octyl, 2,3-dimethylhexyl, and the like.

The term “alkenyl” in the present invention refers to a linear or branched hydrocarbon containing one or multiple double bonds. Alkenyl group can have 2 to 9 carbon atoms, and also includes alkenyl groups with no specified chain length. The alkenyl group can be a medium-sized alkenyl group containing 2 to 9, sometimes preferably 2 to 6, carbon atoms. The alkenyl group can also be a small-sized alkenyl group containing 2 to 4 carbon atoms. The alkenyl group can be designed as “C2-4 alkenyl” or similar designs. For example, “C2-4 alkenyl” means that there are 2-4 carbon atoms in the alkenyl chain, that is, the alkenyl chain can be selected from: ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2-dienyl, buta-1,2-dien-4-yl. Typical alkenyl includes, but not limited to: ethenyl, propenyl, butenyl, pentenyl, hexenyl and the like.

The term “alkynyl” in the present invention refers to a linear or branched hydrocarbon containing one or multiple triple bonds. Alkynyl group can have 2 to 9 carbon atoms, and also includes alkynyl groups with no specified chain length. The alkynyl group can be a medium-sized alkynyl group containing 2 to 9 carbon atoms. The alkynyl group can also be a lower alkynyl group containing 2 to 4 carbon atoms. The alkynyl group can be designed as “C2-4 alkynyl” or similar designs. For example, “C2-4 alkynyl” means that there are 2-4 carbon atoms in the alkynyl chain, that is, the alkynyl chain can be selected from: ethynyl, propyn-1-yl, propyn-2-yl, butyn-1-yl, butyn-2-yl, butyn-3-yl and 2-butynyl. Typical alkynyl includes, but not limited to: ethynyl, propynyl, butynyl, pentynyl, hexynyl and the like.

The term “aryl” in the present invention refers to a ring or ring system with conjugated π-electron system and includes carbocyclic aryl (such as phenyl) and heterocyclic aryl (such as pyridine). The term includes groups with a single ring or multiple fused rings (i.e., rings that share a pair of adjacent atoms), and the whole ring system is aromatic.

The term “heteroaryl” in the present invention refers to an aromatic ring or ring system (i.e., two or multiple fused rings that share two adjacent atoms) containing one or multiple heteroatoms. That is, in addition to carbon, the ring skeleton includes, but not limited to, nitrogen, oxygen, sulfur and other elements. When heteroaryl is a ring system, each ring in the system is aromatic. Heteroaryl can have 5-18 ring members (i.e., the number of atoms constituting the ring skeleton, including the number of carbon atoms and heteroatoms). The current definition also includes heteroaryl groups with no specified ring size. Examples of heteroaryl include, but not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridyl, pyridazinyl, pyrimidinyl, pyrazinyl, triazinyl, quinolinyl, isoquinolinyl, benzimidazolyl, benzoxazolyl, benzothiazolyl, indolyl, isoindolyl, benzothienyl.

The term “cycloalkyl” in the present invention refers to a fully saturated carbocyclic or ring system. Examples include, but not limited to, cyclopropyl, cyclobutyl, cyclopentyl, cyclohexyl.

The term “(heterocyclyl) alkyl” in the present invention refers to heterocyclyl as a substituent connected to other groups through alkylene. Examples include, but not limited to, imidazolinyl methyl and indolinyl ethyl. The term “heterocyclyl” refers to a non-aromatic ring or ring system containing at least one heteroatom in its skeleton. Heterocyclyl can be connected in the form of fused rings, bridged rings or spiro rings. At least one ring in the heterocyclyl ring system is non-aromatic, and it can have any degree of saturation. The heteroatom can be located on the non-aromatic or aromatic ring of the ring system. The heterocyclyl can have 3 to 20 ring atoms (i.e., the number of atoms constituting the ring skeleton, including the number of carbon atoms and heteroatoms). The current definition also includes heterocyclyl groups with no specified range of ring numbers. The heterocyclyl group can be a medium-sized heterocyclyl group containing 3 to 10 ring atoms. The heterocyclyl group can also be a small-sized heterocyclyl group containing 3 to 6 ring atoms. Examples of heterocyclyl include, but not limited to: azepinyl, acridinyl, carbazolyl, cinnolinyl, dioxolanyl, imidazolinyl, imidazolidinyl, morpholinyl, oxiranyl, oxepanyl, thietanyl, piperidinyl, piperazinyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathianyl, 1,4-oxathianyl, 2H-1,3-dioxolanyl, 1,3-dithiolanyl, 1,3-dithiolanyl, isoxazolinyl, isoxazolidinyl, oxazolinyl, oxazolidinyl, oxazolidinone, oxazolidinone, thiazolidinyl, 1,3-oxathiolyl, indolinyl, isoindolinyl, tetrahydrofuranyl, tetrahydropyranyl, tetrahydrothienyl, tetrahydrothiopyranyl, tetrahydro-1,4-thiazinyl, thiomorpholinyl, dihydrobenzofuranyl, benzimidazolidinyl and tetrahydroquinolinyl.

As used herein, “alkoxy” refers to the formula —OR wherein R is an alkyl as is defined above, such as “C1-9 alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkylthio” refers to the formula SR wherein R is an alkyl as is defined above, such as “C1-9 alkylthio” and the like, including but not limited to methylmercapto, ethylmercapto, n-propylmercapto, 1-methylethylmercapto (isopropylmercapto), n-butylmercapto, iso-butylmercapto, sec-butylmercapto, tert-butylmercapto, and the like.

As used herein, “aryloxy” and “arylthio” refers to RO- and RS-, in which R is an aryl as is defined above, such as “C6-10 aryloxy” or “C6-10 arylthio” and the like, including but not limited to phenyloxy.

The term “halogen” or “halo” as used herein, means any one of the radio-stable atoms of column 7 of the Periodic Table of the Elements, e. g., fluorine, chlorine, bromine, or iodine, sometimes with bromine and chlorine being preferred.
“Bond” refers to a covalent bond using a sign of “—”.
“Hydroxy” refers to an —OH group.
“Amino” refers to a —NH₂ group.
“Cyano” refers to a —CN group.
“Nitro” refers to a —NO₂ group.
“Oxo group” refers to a ═O group.
“Carboxyl” refers to a —C(═O)OH group.

Any of the alkyl, alkenyl, alkynyl, aryl, cycloalkyl, heteroaryl, and heterocyclyl groups, whether standalone or being a part of another group, such as alkoxy, alkylthio, aryloxy, or the like, may be substituted or unsubstituted. When substituted, the substituents group(s) can be substituted at any available connection point, and the substituent group(s) can be one or more, sometimes preferably one to five, and sometimes more preferably one to three, groups independently selected from halogen, C₁-C₆ alkyl, C₁-C₆ haloalkyl, C₁-C₆ alkoxy, C₂-C₆ alkenyl, C₂-C₆ alkynyl, C₁-C₆ alkylsulfo, C₁-C₆ alkylamino, thiol, hydroxy, nitro, cyano, amino, C₃-C₆ cycloalkyl, 5- to 10-membered heterocyclyl, C₆-C₁₀ aryl, 5- to 10-membered heteroaryl, and oxo groups, or the like.

“Optional” or “optionally” means that the event or circumstance described subsequently can, but need not, occur, and the description includes the instances in which the event or circumstance may or may not occur. For example, “the heterocyclic group optionally substituted by an alkyl” means that an alkyl group can be, but need not be, present, and the description includes the case of the heterocyclic group being substituted with an alkyl and the heterocyclic group being not substituted with an alkyl. The phrases “optionally substituted” and “substituted or unsubstituted” are sometimes used interchangeably.

“Substituted” refers to one or more hydrogen atoms in the group, preferably up to 5, more preferably 1 to 3 hydrogen atoms, independently substituted with a corresponding number of substituents. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

A “pharmaceutical composition” refers to a mixture of one or more of the compounds described in the present disclosure or physiologically/pharmaceutically acceptable salts or prodrugs thereof and other chemical components such as physiologically/pharmaceutically acceptable carriers and excipients. The purpose of a pharmaceutical composition is to facilitate administration of a compound to an organism, which is conducive to the absorption of the active ingredient and thus displaying biological activity.

“Pharmaceutically acceptable salts” refer to salts of the compounds of the disclosure, such salts being safe and effective when used in a mammal and have corresponding biological activity. The salts can be prepared during the final isolation and purification of the compounds or separately by reacting a suitable nitrogen atom with a suitable acid. Acids commonly employed to form pharmaceutically acceptable salts include inorganic acids such as hydrochloric acid, hydrobromic acid, hydroiodic acid, sulfuric acid, phosphoric acid, hydrogen bisulfide as well as organic acids, such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, acetic acid acid, and related inorganic and organic acids.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include, but are not limited to, lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, and N-methylmorpholine.

The term “pharmaceutically acceptable,” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

The term “therapeutically effective amount,” as used herein, refers to the total amount of each active component that is sufficient to show a meaningful patient benefit, e.g., a sustained reduction in viral load. When applied to an individual active ingredient, administered alone, the term refers to that ingredient alone. When applied to a combination, the term refers to combined amounts of the active ingredients that result in the therapeutic effect, whether administered in combination, serially, or simultaneously.

The term “treat”, “treating”, “treatment”, or the like, refers to: (i) inhibiting the disease, disorder, or condition, i.e., arresting its development; and (ii) relieving the disease, disorder, or condition, i.e., causing regression of the disease, disorder, and/or condition. In addition, the compounds of present disclosure may be used for their prophylactic effects in preventing a disease, disorder or condition from occurring in a subject that may be predisposed to the disease, disorder, and/or condition but has not yet been diagnosed as having it.

As used herein, the singular forms “a”, “an”, and “the” include plural reference, and vice versa, unless the context clearly dictates otherwise.

When the term “about” is applied to a parameter, such as pH, concentration, temperature, or the like, it indicates that the parameter can vary by +10%, and sometimes more preferably within 55%. As would be understood by a person skilled in the art, when a parameter is not critical, a number is often given only for illustration purpose, instead of being limiting.

### Abbreviations

As used herein, common organic abbreviations are defined as follows:
Ac Acetyl
ACN Acetonitrile
Ala Alanine
Asn Asparagine
aq. Aqueous
BOC or Boc tert-Butoxycarbonyl
BSA Bovine Serum Albumin
° C. Temperature in degrees Centigrade
Cit Citrulline
DCM dichloromethane
DIEA Diisopropylethylamine
DMF N,N-Dimethylformamide
EDC 1-Ethyl-3-(3-dimethylaminopropyl)carbodiimide
Et Ethyl
EtOAc Ethyl acetate
Eq Equivalent
Fmoc 9-Fluorenylmethoxycarbonyl
g Gram(s)
GSH Glutathione
h Hour (hours)
HATU 2-(1H-7-azabenzotriazol-1-yl)-1,1,3,3-tetramethyl uranium
hexafluorophosphate
HOBt N-Hydroxybenzotriazole
HPLC High-performance liquid chromatography
KLH Keyhole Limpet Hemocyanin
LC/MS Liquid chromatography-mass spectrometry
Lys Lysine
Me Methyl
mg milligram(s)
MeOH Methanol
mL Milliliter(s)
mL, uL Microliter(s)
mol mole(s)
mmol millimole(s)
mmol, umol micromole(s)
MS mass spectrometry
NHS N-Hydroxysuccinimide
OVA Ovalbumin
PAB p-aminobenzyl
Pip piperidine
PyBOP benzotriazol-1-yloxytripyrrolidinophosphonium hexafluorophosphate
RP-HPLC reverse phase HPLC
RT/rt room temperature
t-Bu tert-Butyl
Tert, t tertiary
TFA Trifluoracetic acid
THF Tetrahydrofuran
Val Valine

### Synthetic Methods

### Synthesis and Preparation Methods

Compounds of formular I and II may be produced by processes known to those skilled in the art by following the reactions in Scheme 1 and Scheme 2, and example section.

Preparation of SBC scaffold VI and its disubstituted alternatives IX
Synthesis of the core scaffold IX (Scheme 1)

From commercially available starting materials, such as di-tert-butyl hydrazine-1,2-dicarboxylate, 3,4-dibromofuran-2,5-dione (IV), and 4,5-dibromo-1,2-dihydropyridazine-3,6-dione (I), cyclic alkyl 4,5-dibromo-1,2-dihydropyridazine-3,6-dione scaffold IX can be prepared in 1-3 steps. Treating dibromo precursors (II) bearing a functional group (e.g. carboxylate group) with bis-Boc protected hydrazine produced Boc-protected hydrazine V. Next, heating intermediate V with 3,4-dibromofuran-2,5-dione (IV) or furan-2,5-dione (VII) afford cycloalkyl dibromo pyridazinedione scaffold VI and pyridazinedione scaffold VIII respectively. Dibromo pyridazinedione scaffold VI could further react with thiols and phenols to produce derivatives IX. Compound VI and IX both are readily for further derivatization (shown in Scheme 2). In an alternative fashion, 4,5-dibromo-1,2-dihydropyridazine-3,6-dione (I) can be directly alkylated by dibromo alkyl ester II to afford the dibromo pyridazinedione scaffold III as a t-butyl ester.

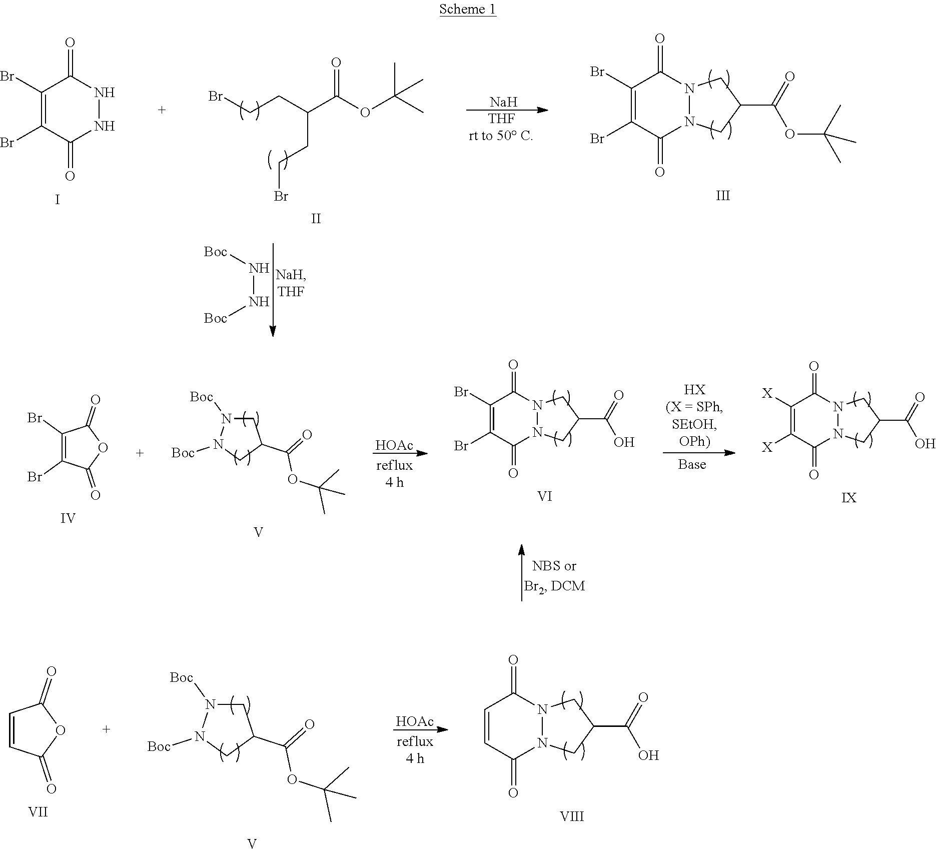

Functionalization and derivatization of disubstituted cycloalkyl pyridazinedione scaffold IX
(1) Functionalization of the core scaffold IX (Scheme 2)

A common method for adding functionality to rebridging scaffold IX is to couple a spacer linker R¹ bearing an amine functional group using a variety of amide coupling reagents such as DCC, EDCI, HATU or PyBOP, or via an activated HOSu ester. The resultant amide bond shows excellent stability in vivo, and a great deal of toxic payloads, fluorescent dyes, and imaging agents are commercially available as amines. The functionalized intermediate X then can selectively react with alkyl thiols, arylthiols, cysteine, GSH, or thiols derived from the reduced disulfide bonds of proteins and antibodies. Different nucleophilic compounds can be introduced sequentially to afford monosubstituted product XI and disubstituted product XIII. Furthermore, the functionalized intermediate X is ready to form disulfide bonds to rebridge the thiol groups from peptides or derived from reduced antibodies.

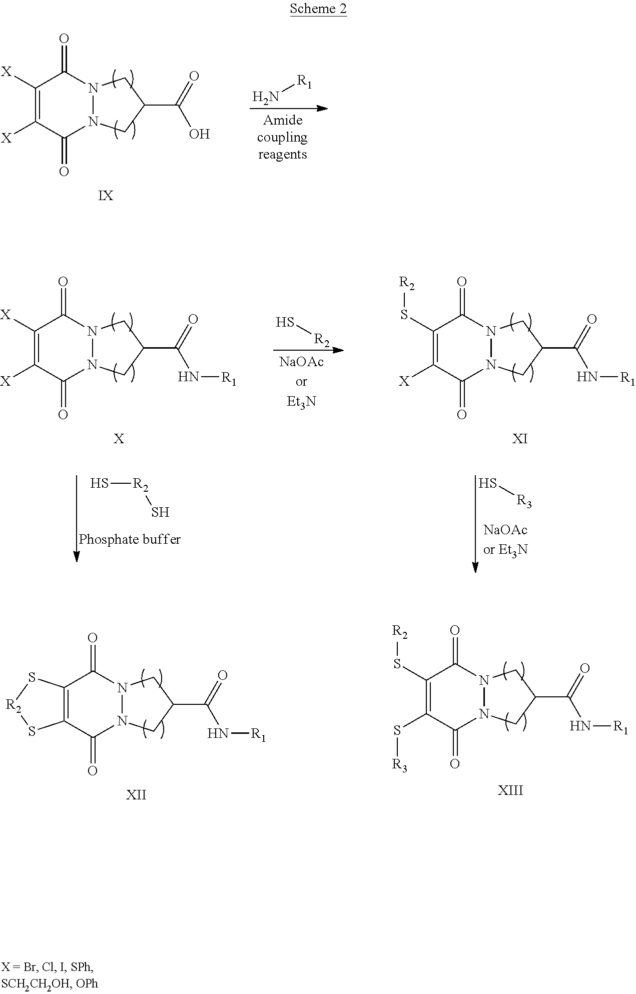

### EXAMPLES

The present invention is further exemplified, but not limited, by the following Examples, which illustrate certain aspects of the invention, including preparation of compounds.

### Example 1

### Synthesis of tritert-butyl diazepane-1,2,5-tricarboxylate (E01)

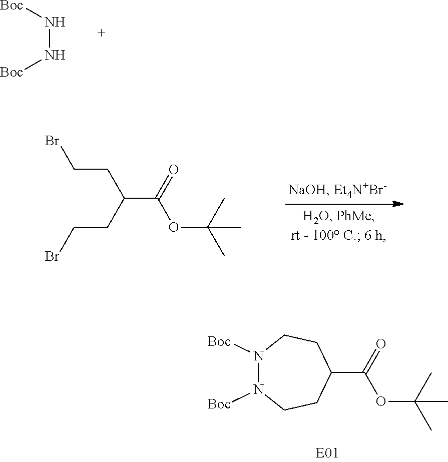 [SMILES: CC(C)(C)OC(=O)NNC(=O)OC(C)(C)C ; CC(C)(C)OC(=O)C(CCBr)CCBr ; CC(C)(C)OC(=O)C1CCN(C(=O)OC(C)(C)C)N(C(=O)OC(C)(C)C)CC1]

A two phase reaction mixture of di-tert-butyl hydrazine-1,2-dicarboxylate (2.3 g, 1.0 mmole), TEAB (0.1 g, 0.7 mmol) and tert-butyl 4-bromo-2-(2-bromoethyl)butanoate (5.0, 1.5 mmol) in 2/1 toluene/50% aqueous sodium hydroxide (15 mL) was stirred vigorously and warmed to 100° C. A thick white solid formed. After 6 hours the reaction was allowed to cool to room temperature, diluted with ethyl acetate (50 mL), and the organic phase washed with 10% sodium bicarbonate (20 mL), water (20 mL) and brine (20 mL), dried (Na₂SO₄) and concentrated in vacuo to give 3.4 g of tri-tert-butyl diazepane-1,2,5-tricarboxylate as a white solid (86%). LCMS: 401.3 [M+H⁺].

### Example 2

### Synthesis of tert-butyl 2-[[tert-butoxycarbonyl-(tert-butoxycarbonylamino)amino]methyl]prop-2-enoate (3)

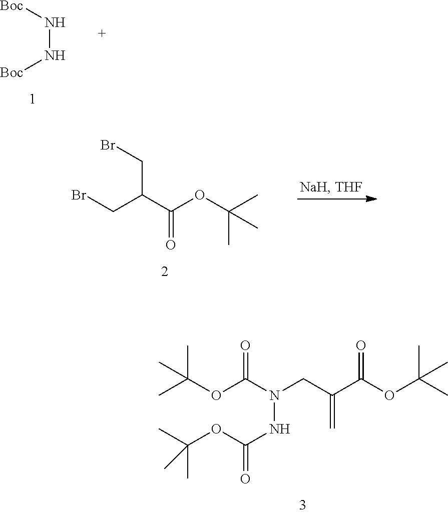 [SMILES: CC(C)(C)OC(=O)NNC(=O)OC(C)(C)C ; CC(C)(C)OC(=O)C(CBr)CBr ; C=C(CN(NC(=O)OC(C)(C)C)C(=O)OC(C)(C)C)C(=O)OC(C)(C)C]

To a solution of di-tert-butyl hydrazine-1,2-dicarboxylate (1) (76.8 mg, 0.33 mmol) and tert-butyl 3-bromo-2-(bromomethyl)propanoate (2) (200 mg, 0.66 mmol) in 3 mL of anhydrous THF was added NaH (60% in oil, 80 mg, 2.0 mmol). The mixture was stirred at room temperature for 15 min and then quenched with a solution of 60 μL of AcOH in 1 mL of water.

Then the mixture was purified by preparative HPLC. The pure fractions were lyophilized to give 204 mg of title compound 3 as a white solid. LCMS: 373.6 [M+H⁺].

### Example 3

### Synthesis of 6,7-dibromo-5,8-dioxo-2,3,5,8-tetrahydro-1H-pyrazolo[1,2-a]pyridazine-2-carboxylic acid (E03)

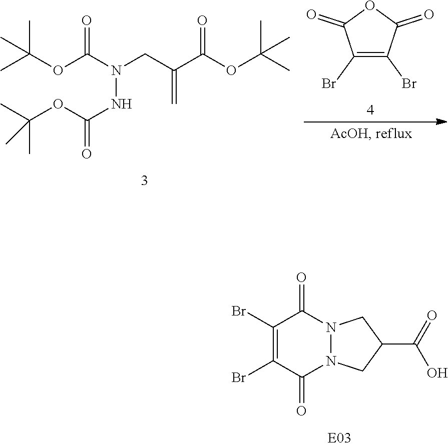

To a solution of tert-butyl 2-[[tert-butoxycarbonyl-(tert-butoxycarbonylamino)amino]methyl]prop-2-enoate (3) (204 mg, 0.55 mmol) in 8.0 mL of glacial AcOH was added 3,4-dibromofuran-2,5-dione (4) (140 mg, 0.55 mmol). The mixture was stirred under reflux under argon gas atmosphere for 11 days, then concentrated to 3 mL and purified by preparative HPLC. The pure fractions were lyophilized to give 43 mg of the title compound E03 as a white solid (22%). LCMS: 354.8 [M+H⁺].

### Example 4

### Synthesis of 5,8-dioxo-2,3,5,8-tetrahydro-1H-pyrazolo[1,2-a]pyridazine-2-carboxylic acid (E02)

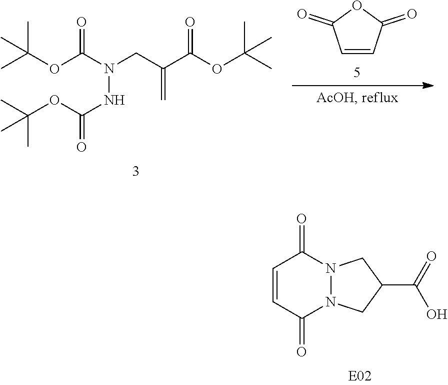

To a solution of tert-butyl 2-[[tert-butoxycarbonyl-(tert-butoxycarbonylamino)amino]methyl]prop-2-enoate (3) (372 mg, 1.0 mmol) in 15 mL of glacial AcOH was added furan-2,5-dione (5) (98 mg, 1.0 mmol). The mixture was stirred under reflux under argon gas atmosphere for 7 days, then concentrated to 3 mL and purified by preparative HPLC. The pure fractions were lyophilized to give 136 mg of the title compound E02 as a white solid (69%). LCMS: 197.2 [M+H⁺].

### Example 5

### Synthesis of 6,7-dibromo-5,8-dioxo-2,3,5,8-tetrahydro-1H-pyrazolo[1,2-a]pyridazine-2-carboxylic acid (E03)

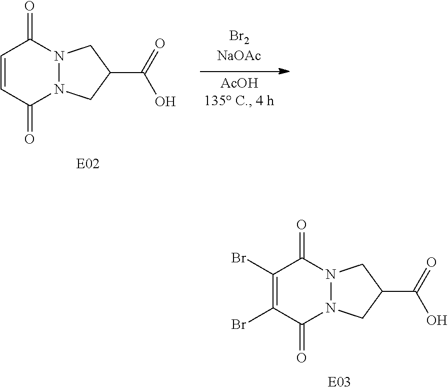 [SMILES: O=C(O)C1Cn2c(=O)ccc(=O)n2C1 ; O=C(O)C1Cn2c(=O)c(Br)c(Br)c(=O)n2C1]

5,8-Dioxo-2,3,5,8-tetrahydro-1H-pyrazolo[1,2-a]pyridazine-2-carboxylic acid (186 mg; 0.95 mmol; 1.0 eq) and sodium acetate (171 mg; 2.08 mmol; 2.2 eq) were solubilized in acetic acid (2.7 mL) in a tube at 0° C. Bromine (107 μL, 2.08 mmol, 2.2 eq) was added, the tube was sealed, and the mixture was stirred at 135° C. for 4 h. After cooling down to 0° C., water (10 mL) was added, and the solution was extracted with ethyl acetate (3×15 mL).

Organic phases were combined, washed with sodium thiosulfate (2×15 mL), dried over magnesium sulfate, and concentrated under reduced pressure. Residual acetic acid was co-evaporated with toluene under reduced pressure. After purification by flash chromatography (SiO₂, cyclohexane/ethyl acetate, 40:60), 6,7-dibromo-5,8-dioxo-2,3,5,8-tetrahydro-1H-pyrazolo[1,2-a]pyridazine-2-carboxylic acid (273 mg; 78%) was obtained as a white solid. LCMS: 354.8 [M+H⁺].

### Example 6

### Synthesis of tert-butyl 6,7-dibromo-5,8-dioxo-2,3-dihydro-1H-pyrazolo[1,2-a]pyridazine-2-carboxylate (E04)

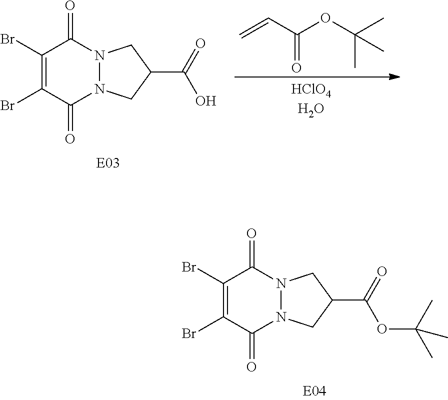

The title compound was prepared by treating 6,7-dibromo-5,8-dioxo-2,3,5,8-tetrahydro-1H-pyrazolo[1,2-a]pyridazine-2-carboxylic acid with t-butylacrylate (25 eq) at 25° C. for 48 h in a stoppered flask containing 3 drops of 60% HClO₄, following by a careful neutralization (10% NaHCO₃), extraction (dichloromethane, three times), and subsequent drying and evaporation of the organic phase (86%). LCMS: 408.9 [M+H⁺].

### Example 7

### Synthesis of tert-butyl 6-bromo-5,8-dioxo-7-phenylsulfanyl-2,3-dihydro-1H-pyrazolo[1,2-a]pyridazine-2-carboxylate (6)

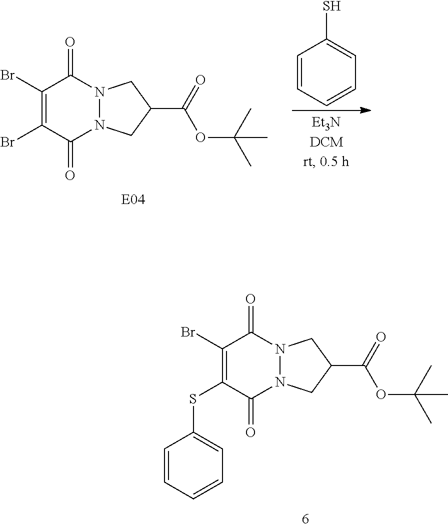 [SMILES: CC(C)(C)OC(=O)C1Cn2c(=O)c(Br)c(Br)c(=O)n2C1 ; Sc1ccccc1 ; CC(C)(C)OC(=O)C1Cn2c(=O)c(Br)c(Sc3ccccc3)c(=O)n2C1]

To a solution of thiophenol (0.05 mL, 0.5 mmol) and triethylamine (0.18 mL, 1.3 mmol) in dichloromethane (6 mL) at 21° C., was added a solution of tert-butyl 6,7-dibromo-5,8-dioxo-2,3-dihydro-1H-pyrazolo[1,2-a]pyridazine-2-carboxylate (185 mg, 0.45 mmol) in dichloromethane (6 mL), and the reaction mixture was stirred for 30 min. Following this, the reaction mixture was diluted with dichloromethane (20 mL) and washed with water (3×15 mL) and brine (15 mL). The organic phase was dried over MgSO4, concentrated in vacuo and the crude residue was purified by flash column chromatography (15-85% EtOAc/Hexanes). The appropriate fractions were then combined and concentrated in vacuo to afford tert-butyl 6-bromo-5,8-dioxo-7-phenylsulfanyl-2,3-dihydro-1H-pyrazolo[1,2-a]pyridazine-2-carboxylate (176 mg, 0.40 mmol, 89%) as a green solid. LRMS: 439.0 [M+H⁺].

### Example 8

### Synthesis of tert-butyl 5,8-dioxo-6,7-bis(phenylsulfanyl)-2,3-dihydro-1H-pyrazolo[1,2-a]pyridazine-2-carboxylate (E07)

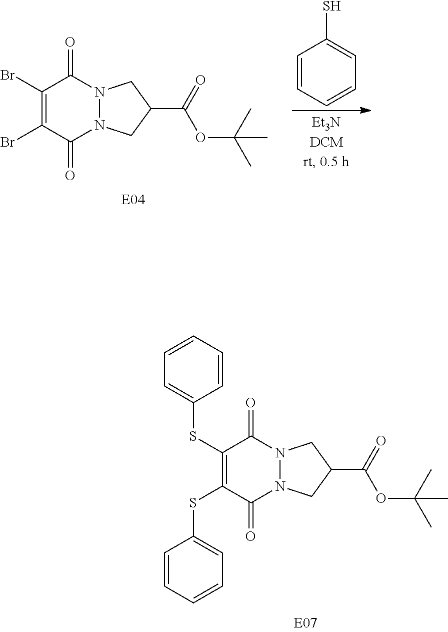 [SMILES: CC(C)(C)OC(=O)C1Cn2c(=O)c(Br)c(Br)c(=O)n2C1 ; Sc1ccccc1 ; CC(C)(C)OC(=O)C1Cn2c(=O)c(Sc3ccccc3)c(Sc3ccccc3)c(=O)n2C1]

To a solution of thiophenol (0.10 mL, 0.97 mmol) and triethylamine (0.39 mL, 2.80 mmol) in dichloromethane (6 mL) at 25° C., was added a solution of tert-butyl 6,7-dibromo-5,8-dioxo-2,3-dihydro-1H-pyrazolo[1,2-a]pyridazine-2-carboxylate (127 mg, 0.31 mmol) in dichloromethane (6 mL), and the reaction mixture was stirred for 30 min. Following this, the reaction mixture was diluted with dichloromethane (20 mL) and washed with water (3×15 mL) and brine (15 mL). The organic phase was dried over MgSO₄, concentrated in vacuo and the crude residue was purified by flash column chromatography (15-80% EtOAc/Hexanes). The appropriate fractions were then combined to afford tert-butyl 5,8-dioxo-6,7-bis(phenylsulfanyl)-2,3-dihydro-1H-pyrazolo[1,2-a]pyridazine-2-carboxylate (108 mg, 0.23 mmol, 76%) as a yellow solid. LCMS: 469.1 [M+H⁺].

### Example 9

### tert-butyl 6,7-bis(2-hydroxyethylsulfanyl)-5,8-dioxo-2,3-dihydro-1H-pyrazolo[1,2-a]pyridazine-2-carboxylate (E05)

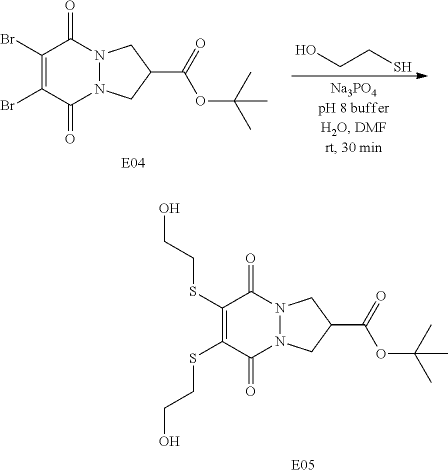

To 2-mercaptoethanol (70 ul, 1 mmol) in buffer (10 ml, 150 mM NaCl, 100 mM sodium phosphate, pH 8.0, 5.0% DMF) was added tert-butyl 6,7-dibromo-5,8-dioxo-2,3-dihydro-1H-pyrazolo[1,2-a]pyridazine-2-carboxylate (160 mg, 0.39 mmol) in DMF (0.25 ml). The reaction was stirred for 30 min at RT and lithium chloride (2 g) was added. The aqueous reaction mixture was extracted with ethyl acetate (7×15 ml). The organic layers were combined, the solvent removed in vacuo and the residual material was purified by flash chromatography on silica gel (hexanes: ethyl acetate 1:1 to 1:9). Fractions containing the product were collected and the solvent were removed in vacuo to afford the title compound as a yellow solid (83 mg, 53%). LCMS: 405.1 [M+H⁺].

### Example 10

### Synthesis of tert-butyl 6,7-bis[[(2R)-3-methoxy-2-(methylamino)-3-oxo-propyl]sulfanyl]-5,8-dioxo-2,3-dihydro-1H-pyrazolo[1,2-a]pyridazine-2-carboxylate (E09)

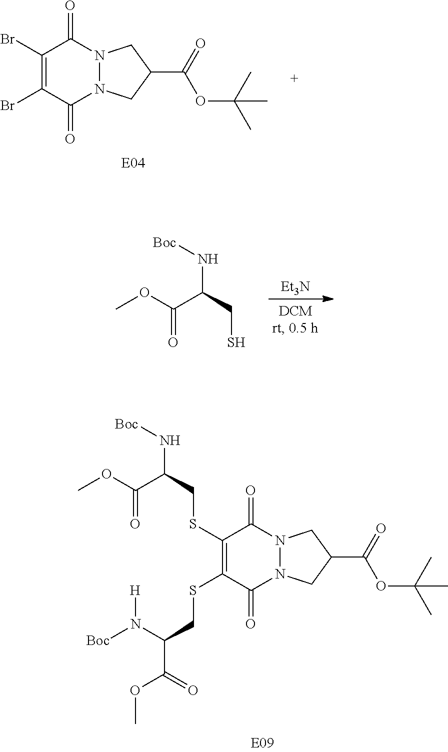 [SMILES: "Et3N" ; CC(C)(C)OC(=O)C1Cn2c(=O)c(Br)c(Br)c(=O)n2C1 ; COC(=O)[C@H](CS)NC(=O)OC(C)(C)C ; COC(=O)[C@H](CSc1c(SC[C@H](NC(=O)OC(C)(C)C)C(=O)OC)c(=O)n2n(c1=O)CC(C(=O)OC(C)(C)C)C2)NC(=O)OC(C)(C)C]

To a solution of tert-butyl 6,7-dibromo-5,8-dioxo-2,3-dihydro-1H-pyrazolo[1,2-a]pyridazine-2-carboxylate (0.16 g, 0.40 mmol) and N-(tert-butoxycarbonyl)-L-cysteine methyl ester (0.47 g, 2.0 mmol) in dichloromethane (10 mL) was added triethylamine (0.07 mL, 0.5 mmol), and the reaction mixture was stirred at 25° C. for 65 h. Following this, the reaction mixture was diluted with dichloromethane (20 mL) and washed with water (3×20 mL) and brine (15 mL). The organic phase was dried over MgSO₄, concentrated in vacuo and the crude residue was purified by flash column chromatography (15-60% EtOAc/Hexanes). The appropriate fractions were then combined and concentrated in vacuo to afford (0.13 g, 0.24 mmol, 59%) as a yellow oil. LCMS: 547.2 [M+H⁺].

### Example 11

### Synthesis of tert-butyl 6-bromo-7-[(2R)-3-methoxy-2-(methylamino)-3-oxo-propyl]sulfanyl-5,8-dioxo-2,3-dihydro-1H-pyrazolo[1,2-a]pyridazine-2-carboxylate (7)

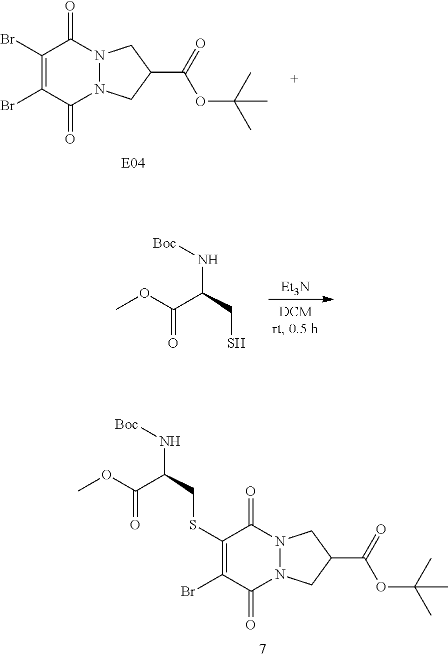 [SMILES: "Et3N" ; CC(C)(C)OC(=O)C1Cn2c(=O)c(Br)c(Br)c(=O)n2C1 ; COC(=O)[C@H](CS)NC(=O)OC(C)(C)C ; COC(=O)[C@H](CSc1c(Br)c(=O)n2n(c1=O)CC(C(=O)OC(C)(C)C)C2)NC(=O)OC(C)(C)C]

To a solution of tert-butyl 6,7-dibromo-5,8-dioxo-2,3-dihydro-1H-pyrazolo[1,2-a]pyridazine-2-carboxylate (0.25 g, 0.61 mmol) in dichloromethane (6 mL) at 25° C., was added dropwise over 30 min a solution of N-(tertbutoxycarbonyl)-L-cysteine methyl ester (0.14 g, 0.59 mmol) and triethylamine (0.13 mL, 0.93 mmol) in dichloromethane (6 mL). Following this, the reaction mixture was diluted with dichloromethane (12 mL) and washed with water (3×15 mL) and brine (15 mL). The organic phase was dried over MgSO₄, concentrated in vacuo and the crude residue was purified by flash column chromatography (0-30% EtOAc/Hexanes). The appropriate fractions were then combined and concentrated in vacuo to afford the title compound (0.17 g, 0.35 mmol, 60%) as a yellow oil. LCMS: 478.1 [M+H⁺].

### Example 12

### Synthesis of tert-butyl 7-[(2R)-3-methoxy-2-(methylamino)-3-oxo-propyl]sulfanyl-5,8-dioxo-6-phenylsulfanyl-2,3-dihydro-1H-pyrazolo[1,2-a]pyridazine-2-carboxylate (E08)

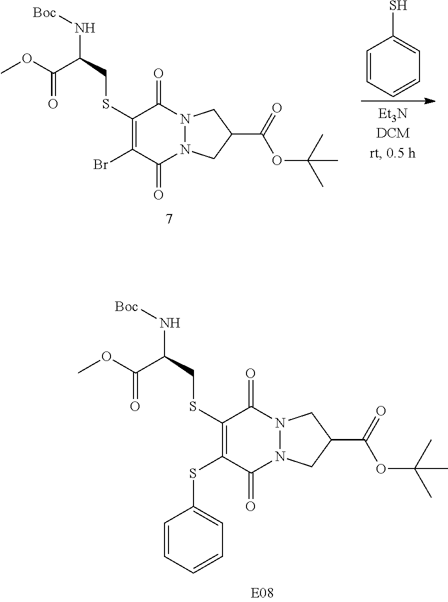

To a solution of tert-butyl 6-bromo-7-[(2R)-3-methoxy-2-(methylamino)-3-oxo-propyl]sulfanyl-5,8-dioxo-2,3-dihydro-1H-pyrazolo[1,2-a]pyridazine-2-carboxylate (0.45 g, 0.94 mmol) in dichloromethane (8 mL) at 25° C., was added a solution of thiophenol (0.10 mL, 0.98 mmol) and triethylamine (0.20 mL, 1.4 mmol) in CH₂Cl₂ (8 mL), and the reaction was stirred for 30 min. Following this, the reaction mixture was diluted with dichloromethane (16 mL) and washed with water (3×15 mL) and brine (15 mL). The organic phase was dried over MgSO4, concentrated in vacuo and the crude residue was purified by flash column chromatography (0-40% EtOAc/Hexanes). The appropriate fractions were then combined and concentrated in vacuo to afford the title compound (0.24 g, 0.47 mmol, 50%) as a yellow oil. LCMS: 508.2 [M+H⁺].

### Example 13

### Synthesis of ethyl 4-[(6,7-dibromo-5,8-dioxo-2,3-dihydro-1H-pyrazolo[1,2-a]pyridazine-2-carbonyl)amino]butanoate (8)

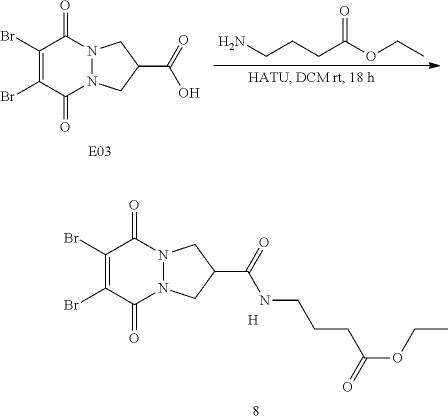

Under inert atmosphere, 6,7-dibromo-5,8-dioxo-2,3-dihydro-1H-pyrazolo[1,2-a]pyridazine-2-carboxylic acid (212 mg, 0.6 mmol) was dissolved in dichloromethane (10 ml). Then, hexafluorophosphate azabenzotriazole tetramethyl uranium (HATU; 300 mg, 0.8 mmol) and 2,6-lutidine (167 μL, 1.4 mmol) were added and the mixture was stirred at room temperature for 10 min. Ethyl 4-aminobutanoate (105 mg, 0.8 mmol) was added and the resulting solution was stirred at room temperature for 18 h before dilution with dimethylsulfoxide and purification by HPLC to give ethyl 4-[(6,7-dibromo-5,8-dioxo-2,3-dihydro-1H-pyrazolo[1,2-a]pyridazine-2-carbonyl)amino]butanoate (227 mg, 81%) as a yellow solid. LCMS: 466.0 [M+H⁺].

### Example 14

### Synthesis of 4-[(6,7-dibromo-5,8-dioxo-2,3-dihydro-1H-pyrazolo[1,2-a]pyridazine-2-carbonyl)amino]butanoic acid (E06)

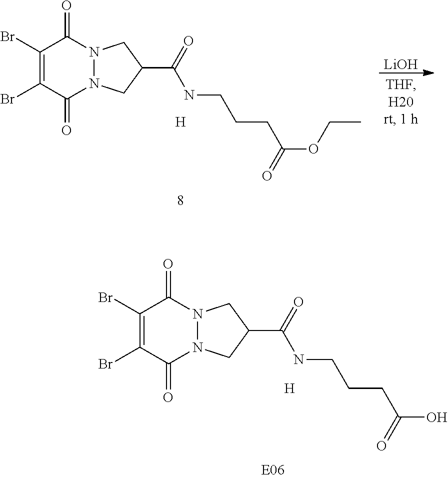

To a solution of ethyl 4-[(6,7-dibromo-5,8-dioxo-2,3-dihydro-1H-pyrazolo[1,2-a]pyridazine-2-carbonyl)amino]butanoate (233 mg, 0.5 mmol, 1 eq) in THF (5 mL) was added LiOH·H₂O (42 mg, 1 mmol, 2 eq) in water (0.5 mL). The mixture was stirred at 25° C. for 1 h. The reaction mixture was concentrated under reduced pressure to give a residue. The residue was purified by prep-HPLC (ACN/H₂O condition) to afford 4-[(6,7-dibromo-5,8-dioxo-2,3-dihydro-1H-pyrazolo[1,2-a]pyridazine-2-carbonyl)amino]butanoic acid (176 mg, 80% yield) as a light-yellow solid. LCMS: 437.9 (M+H+).

### Example 15

### Synthesis of (2,5-dioxopyrrolidin-1-yl) 4-[(6,7-dibromo-5,8-dioxo-2,3-dihydro-1H-pyrazolo[1,2-a]pyridazine-2-carbonyl)amino]butanoate (E10)

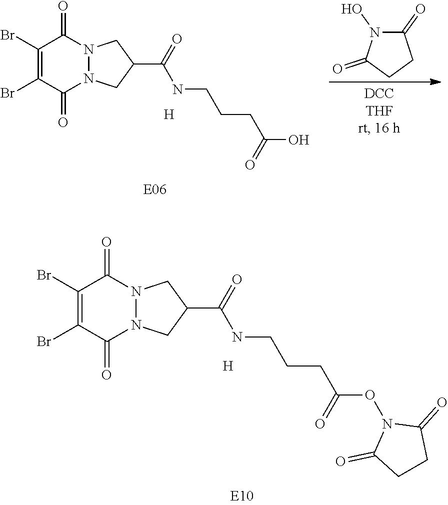

To a solution of 4-[(6,7-dibromo-5,8-dioxo-2,3-dihydro-1H-pyrazolo[1,2-a]pyridazine-2-carbonyl)amino]butanoic acid (307 mg, 0.7 mmol) in THF (10 mL) cooled to 0° C., was added N,N′-dicyclohexylcarbodiimide (160 mg, 0.8 mmol). The homogenous solution was then stirred at 0° C. for 30 min. After this time, was added N-hydroxysuccinimide (89.0 mg, 0.8 mmol) and the reaction stirred at 25° C. for a further 16 h. The newly formed heterogeneous mixture was then filtered and the filtrate concentrated in vacuo. Purification of the crude residue by flash column chromatography (20% to 100% EtOAc/Hexanes) afforded (2,5-dioxopyrrolidin-1-yl) 4-[(6,7-dibromo-5,8-dioxo-2,3-dihydro-1H-pyrazolo[1,2-a]pyridazine-2-carbonyl)amino]butanoate (268 mg, 72%) as a yellow solid. LCMS: 535 [M+H⁺].

### Example 16

### Synthesis of 6,7-dibromo-5,8-dioxo-N-[4-oxo-4-[(2-propylthiazolo[4,5-c]quinolin-4-yl)amino]butyl]-2,3-dihydro-1H-pyrazolo[1,2-a]pyridazine-2-carboxamide (E11, SBC linker-CL-075 payload)

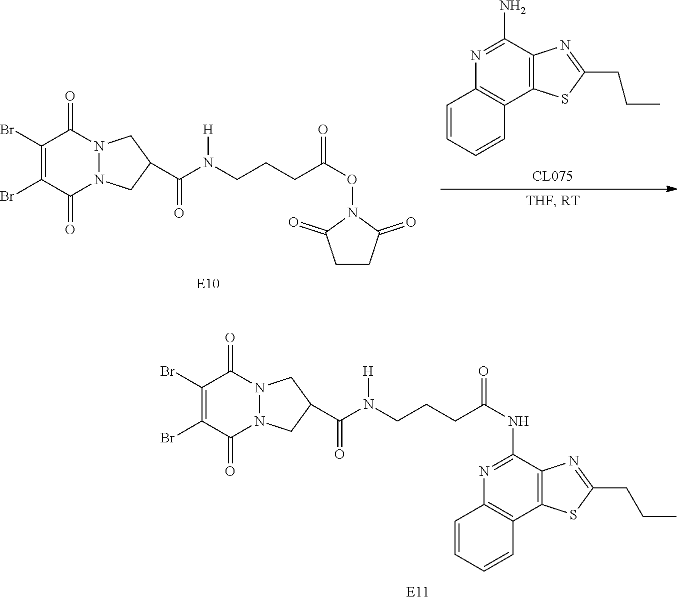 [SMILES: O=C(CCCNC(=O)C1Cn2c(=O)c(Br)c(Br)c(=O)n2C1)ON1C(=O)CCC1=O ; CCCc1nc2c(N)nc3ccccc3c2s1 ; CCCc1nc2c(NC(=O)CCCNC(=O)C3Cn4c(=O)c(Br)c(Br)c(=O)n4C3)nc3ccccc3c2s1]

To a solution of (2,5-dioxopyrrolidin-1-yl) 4-[(6,7-dibromo-5,8-dioxo-2,3-dihydro-1H-pyrazolo[1,2-a]pyridazine-2-carbonyl)amino]butanoate (118 mg, 0.22 mmol) in THF (10 mL), was added 2-propylthiazolo[4,5-c]quinolin-4-amine (innate immune modulator CL-075, 58 mg, 0.24 mmol) and the reaction mixture was stirred at 25° C. for 16 h. After this time, the reaction was concentrated in vacuo and the crude residue dissolved in dichloromethane (50 mL) and washed with water (2×30 mL) and saturated aq. K₂CO₃ (30 mL). The organic layer was then dried (MgSO₄) and concentrated in vacuo. Purification of the crude residue by flash column chromatography (0% to 10% MeOH/EtOAc) afforded the title amide product as a light-yellow solid (102 mg, 70%). LCMS: 663.0 [M+H⁺].

### Example 17

### Antibody/SBC Linker/CL-075 Conjugation (E13)

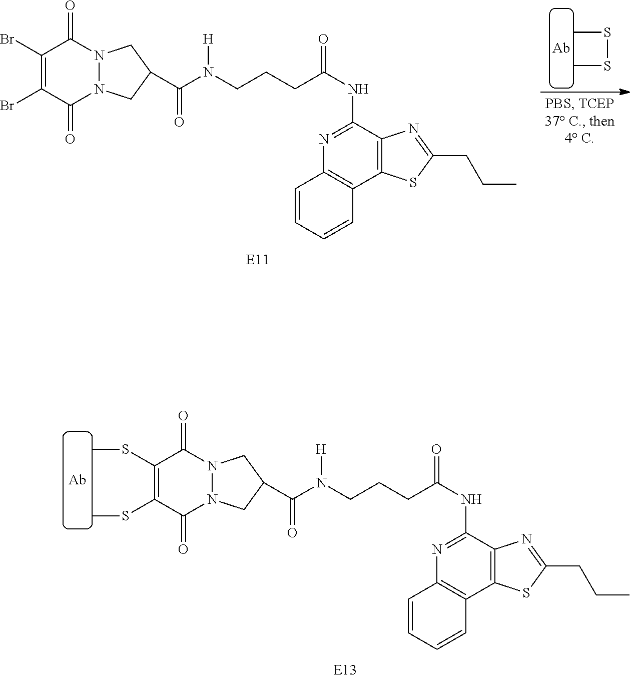

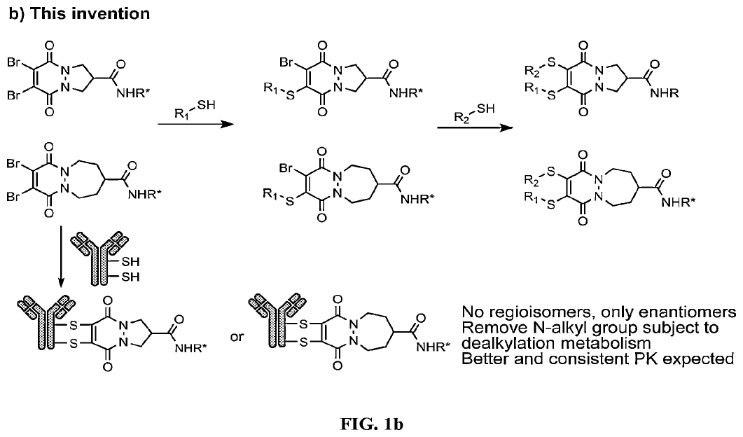

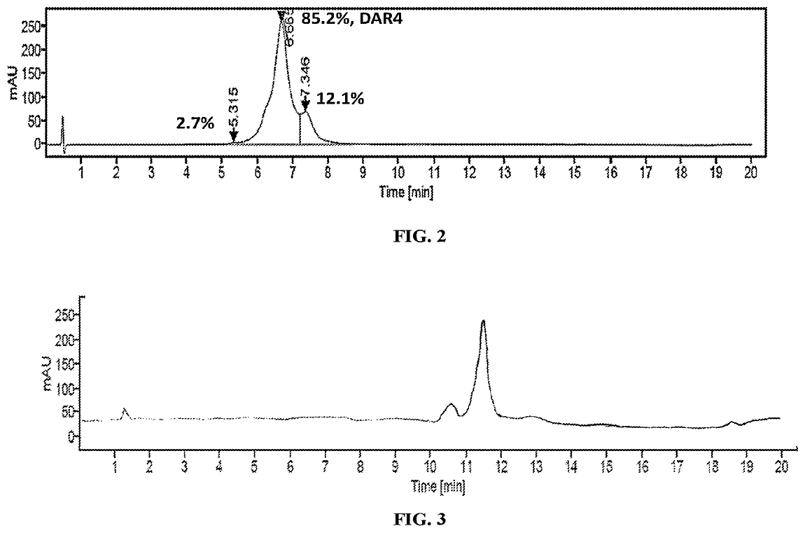

The purified antibody was buffer exchanged into PBS, pH 7.4. 5 eq of Tris (2-carboxyethyl) phosphine (TCEP HCl, 50 mM in deionized water) was freshly prepared and added to a solution of human antibody IgG1 kappa (in house, 5 mg/mL, 1 eq) in Reduction/Conjugation buffer (25 mM Sodium Borate, 25 mM NaCl, 1 mM diethylenetriamine pentaacetate (DTPA) pH 8.0) and the solution incubated at 37° C. for 2 h and cooled to RT. A stock solution of 6,7-dibromo-5,8-dioxo-N-[4-oxo-4-[(2-propylthiazolo[4,5-c]quinolin-4-yl)amino]butyl]-2,3-dihydro-1H-pyrazolo[1,2-a]pyridazine-2-carboxamide (E11, 2 mM in DMSO, 10 eq) was freshly prepared and added and the solution incubated at 4° C. for overnight. Excess reagents were removed by ultrafiltration (6×, 10000 MWCO) into PBS (pH=7.4). The conjugate was characterized by Hydrophobic Interaction Chromatograph-High Performance Liquid Chromatography (HIC-HPLC) (see FIG. 2). An 85% homogenous rate of DAR4 rates was demonstrated.

### Example 18

### Synthesis of SBC Linker-MMAF (E12)

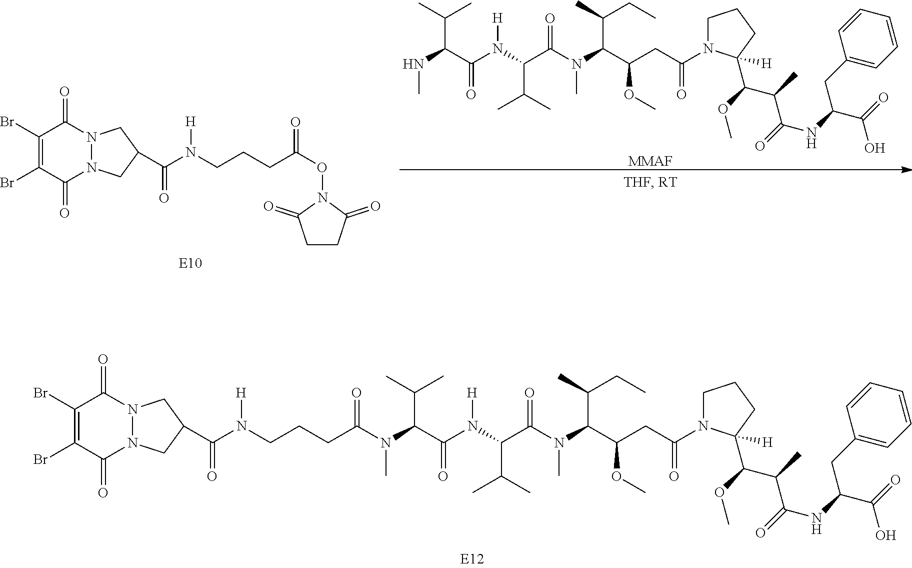

To a solution of (2,5-dioxopyrrolidin-1-yl) 4-[(6,7-dibromo-5,8-dioxo-2,3-dihydro-1H-pyrazolo[1,2-a]pyridazine-2-carbonyl)amino]butanoate (118 mg, 0.22 mmol) in THF (10 mL), was added cytotoxin agent Monomethylauristatin F (MMAF) (161 mg, 0.22 mmol) and the reaction mixture was stirred at 25° C. for 16 h. After this time, the reaction was concentrated in vacuo and the crude residue dissolved in dichloromethane (50 mL) and washed by 10% citric acid aq. (30 mL) and water (2×30 mL) The organic layer was then dried (MgSO₄) and concentrated in vacuo. Purification of the crude residue by prep-HPLC afforded the SBC linker-MMAF payload (E12) as a white solid (165 mg, 65%). LCMS: 1151.4 (M+H+).

### Example 19

### Antibody/SBC Linker/MMAF Conjugation (E14)

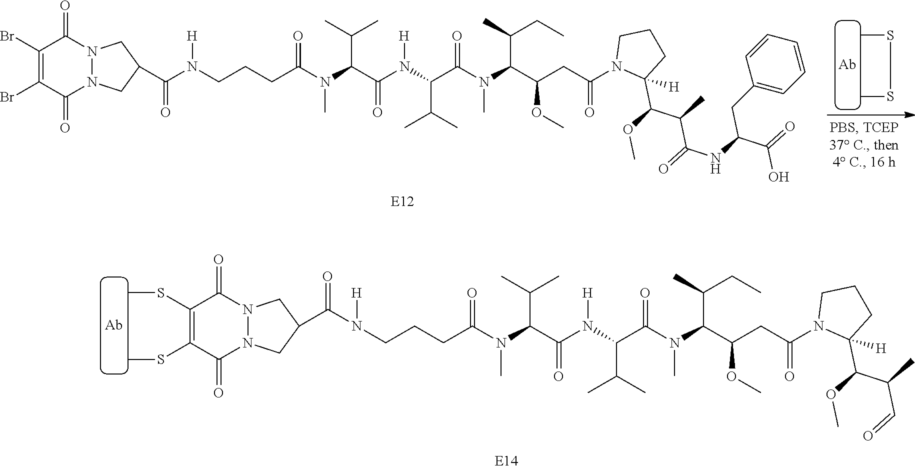

The purified antibody was buffer exchanged into PBS, pH 7.4. 5 eq of TCEP HCl (50 mM in deionized water) was freshly prepared and added to a solution of -human antibody IgG1 kappa (in house, 5 mg/mL, 1 eq) in Reduction/Conjugation buffer (25 mM Sodium Borate, 25 mM NaCl, 1 mM diethylenetriamine pentaacetate (DTPA) pH 8.0) and the solution incubated at 37° C. for 2 h and cooled to RT. A stock solution of SBC linker-MMAF (E12, 2 mM in DMSO, 6 eq) was freshly prepared and added and the solution incubated at 4° C. for overnight. Excess reagents were removed by ultrafiltration (6×, 10000 MWCO) into PBS (pH=7.4). HIC-HPLC diagram showed over 90% of conjugated payload were DAR4 (see FIG. 3).

### Example 20

### Synthesis of 3-(4,5-dibromo-2-methyl-3,6-dioxo-pyridazin-1-yl)-N-(2-propylthiazolo[4,5-c]quinolin-4-yl) propanamide (10, diBrPD-CL075 Payload)

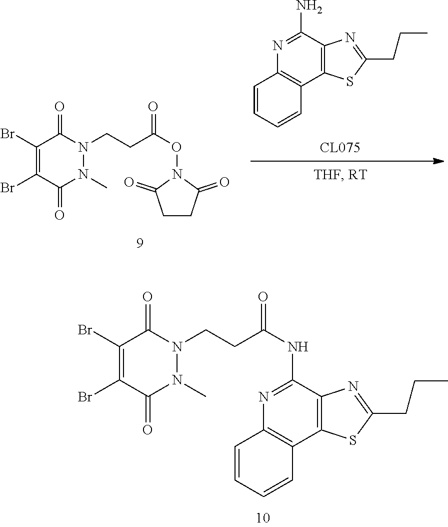 [SMILES: Cn1c(=O)c(Br)c(Br)c(=O)n1CCC(=O)ON1C(=O)CCC1=O ; CCCc1nc2c(N)nc3ccccc3c2s1 ; CCCc1nc2c(NC(=O)CCn3c(=O)c(Br)c(Br)c(=O)n3C)nc3ccccc3c2s1]

2,5-Dioxopyrrolidin-1-yl 3-(4,5-dibromo-2-methyl-3,6-dioxo-3,6-dihydropyridazin-1(2H)-yl) propanoate (9, diBrPD-OSu) was prepared by following the procedure reported in Org. Biomol. Chem., 2018, 16, 1359, supporting information).

To a solution of 2,5-dioxopyrrolidin-1-yl 3-(4,5-dibromo-2-methyl-3,6-dioxo-3,6-dihydropyridazin-1(2H)-yl) propanoate (diBrPD-OSu, 100 mg, 0.22 mmol) in THF (10 mL) was added 2-propylthiazolo[4,5-c]quinolin-4-amine (CL-075, 58 mg, 0.24 mmol) and the reaction mixture was stirred at RT for 16 h. After this time, the reaction was concentrated in vacuo and the crude residue dissolved in dichloromethane (50 mL) and washed with water (2×30 mL) and saturated aq. K₂CO₃ (30 mL). The organic layer was then dried (MgSO₄) and concentrated in vacuo. Purification of the crude residue by flash column chromatography (0% to 10% MeOH/EtOAc) afforded the title amide compound as a light-yellow solid (84 mg, 66%). LCMS: 580.1 [M+H⁺].

### Example 21

### Antibody/diBrPD Linker/CL075 Conjugation (11)

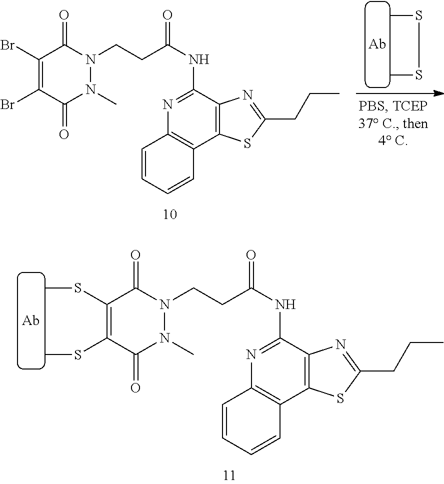

The purified antibody was buffer exchanged into PBS, pH 7.4. 6-10 eq of TCEP HCl (50 mM in deionized water) was freshly prepared and added to a solution of human antibody IgG1 kappa (in house, 5 mg/mL, 1 eq) in Reduction/Conjugation buffer (25 mM Sodium Borate, 25 mM NaCl, 1 mM diethylenetriamine pentaacetate (DTPA) pH 8.0) and the solution incubated at 37° C. for 2 h and cooled to RT. A stock solution of 3-(4,5-dibromo-2-methyl-3,6-dioxo-pyridazin-1-yl)-N-(2-propylthiazolo[4,5-c]quinolin-4-yl)propanamide (diBrPD-CL075 payload, 2 mM in DMSO, 4-20 eq) was freshly prepared and added and the solution incubated at 4° C. for overnight. Excess reagents were removed by ultrafiltration (6×, 10000 MWCO) into PBS (pH=7.4).

TABLE 1
The comparation of homogenous DAR4 rate in a mild condition for a human IgG1 antibody
conjugation with SBC linker-CL075 or diBrPD linker-CL075. Conjugation conditions
showed that antibody-SBC-CL075 used less linker-payload reagent and gained a
higher homogenous DAR4 rate than those with antibody-diBrPD-CL075.

| | | | | Conjugation step 2 | | |
|---|---|---|---|---|---|---|
| Antibody | | Conjugation step 1 | | Linker- | | |
| conjugation | Linker | TCEP/mAb | Reaction | payload/ | Reaction | DAR4 |
| compound | Type | ratio | conditions | mAb ratio | conditions | rate |
| Antibody - SBC- | SBC linker | 6 | 37° C., 2 h | 4 | 4° C., 16 h | 85-90% |
| CL075 (E13) | | | | | | |
| Antibody-diBrPD- | diBrPD | 6 | 37° C., 2 h | 6 | 4° C., 16 h | 56-75% |
| CL075 (11) | linker | | | | | |

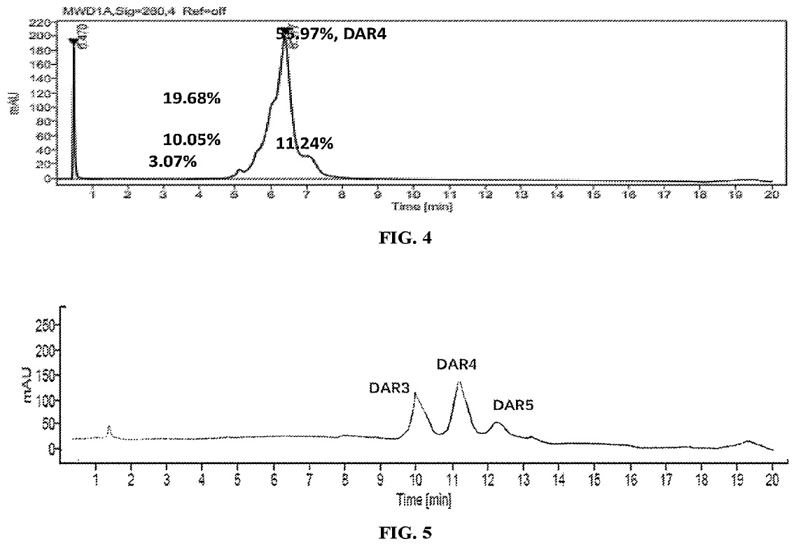

HIC-HPLC diagram demonstrated that using diBrPD linker, human IgG1-CL075 conjugation only obtained the 55.97% of DAR4 homogenous rate, with 19.68% of DAR3, 11.2% of DAR5, 10.05% of DAR2, and 3.07% of DAR1 (see FIG. 4). In contrast, the human IgG1 antibody with the SBC linker in FIG. 2 of Example 17 gained an 85% homogenous rate (see FIG. 2).

### Example 22

### Antibody/diBrPD Linker/MMAF Conjugation (13)

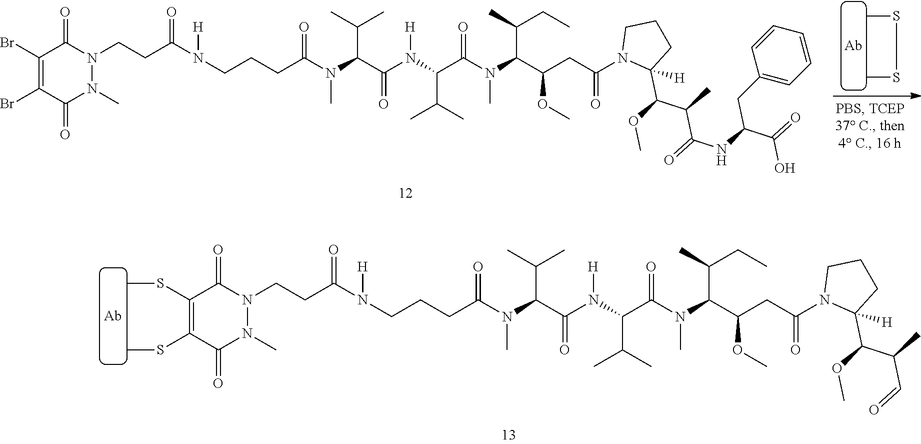

The purified antibody was buffer exchanged into PBS, pH 7.4. 6-10 eq of TCEP·HCl (50 mM in deionized water) was freshly prepared and added to a solution of human antibody IgG1 kappa (5 mg/mL, 1 eq) in Reduction/Conjugation buffer (25 mM Sodium Borate, 25 mM NaCl, 1 mM diethylenetriamine pentaacetate (DTPA) pH 8.0) and the solution incubated at 37° C. for 2 h and cooled to RT. A stock solution of diBrPD-MMAF payload (12, 2 mM in DMSO, 6-8 eq) was freshly prepared and added, then the solution was incubated at 4° C. for overnight. Excess reagents were removed by ultrafiltration (6×, 10000 MWCO) into PBS (pH=−7.4).

HIC-HPLC diagram showed that antibody-diBrPD-MMAF evenly distributed DAR3 and DAR4 with additional DAR5 (see FIG. 5). However, the antibody with the SBC-MMAF in FIG. 3 of Example 19, in comparison, gained a 90% DAR4 homogenous rate.

### Example 23

### Synthesis of 2,5-dioxopyrrolidin-1-yl 6,7-dibromo-5,8-dioxo-2,3,5,8-tetrahydro-1H-pyrazolo[1,2-a]pyridazine-2-carboxylate (E15)

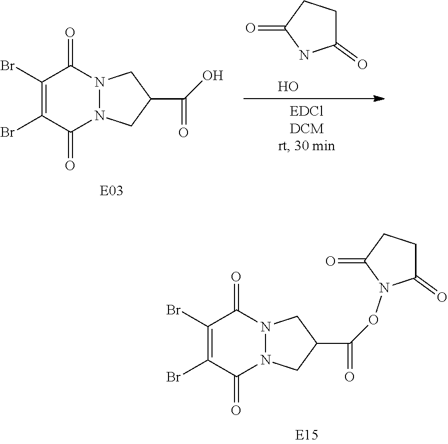

To a stirred solution of 6,7-dibromo-5,8-dioxo-2,3,5,8-tetrahydro-1H-pyrazolo[1,2-a]pyridazine-2-carboxylic acid (350 mg) in anhydrous dichloromethane (10 mL) was added N-hydroxysuccinimide (230 mg), followed by N-(3-dimethylaminopropyl)-N′-ethylcarbodiimide hydrochloride (400 mg). The mixture was stirred at room temperature for 30 min, and the reaction was concentrated to dryness under reduced pressure. The residue was purified directly by RP-HPLC to give the title compound as a white solid (337 mg) after lyophilization. MS found: 452.0 [M+H⁺].

### Example 24

### Synthesis of 4-((S)-2-((S)-2-(3-(4,5-dibromo-2-methyl-3,6-dioxo-3,6-dihydropyridazin-1(2H)-yl)propanamido)-3-methylbutanamido)-5-ureidopentanamido)benzyl (2-propylthiazolo[4,5-c]quinolin-4-yl)carbamate (E16)

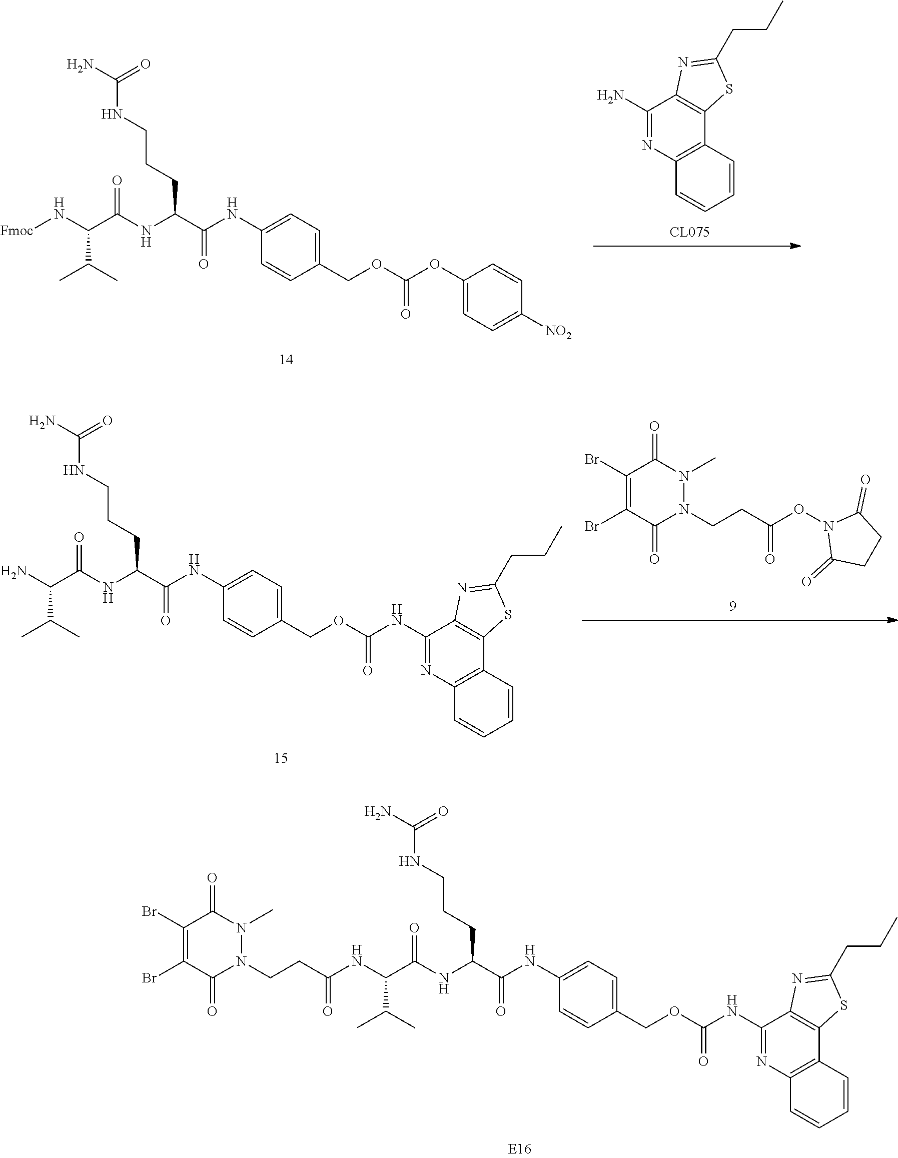

To a solution of compound 14 (40 mg) in anhydrous DMF (1 mL) was added CL075 (10 mg), followed by DIEA (10 mL) and HOBt (2 mg). The reaction mixture was stirred at room temperature (22° C.). After 48 h, piperidine (50 mL) was added and the reaction was stirred at room temperature for 1 h. The mixture was purified directly by RP-HPLC to give compound 15 as a white solid (18 mg) after lyophilization.

To a solution of compound 15 (15 mg) in DMF (1 mL) was added diBrPD-OSu (9) (10 mg), followed by DIEA (8 mL). The reaction mixture was stirred at room temperature. After 3 h, the crude mixture was purified by RP-HPLC to give compound E16 as a white solid (12 mg) after lyophilization. MS found: 987.5 [M+H⁺].

### Example 25

### Synthesis of 4-((S)-2-((S)-2-(6,7-dibromo-5,8-dioxo-2,3,5,8-tetrahydro-1H-pyrazolo[1,2-a]pyridazine-2-carboxamido)-3-methylbutanamido)-5-ureidopentanamido)benzyl (2-propylthiazolo[4,5-c]quinolin-4-yl)carbamate (17)

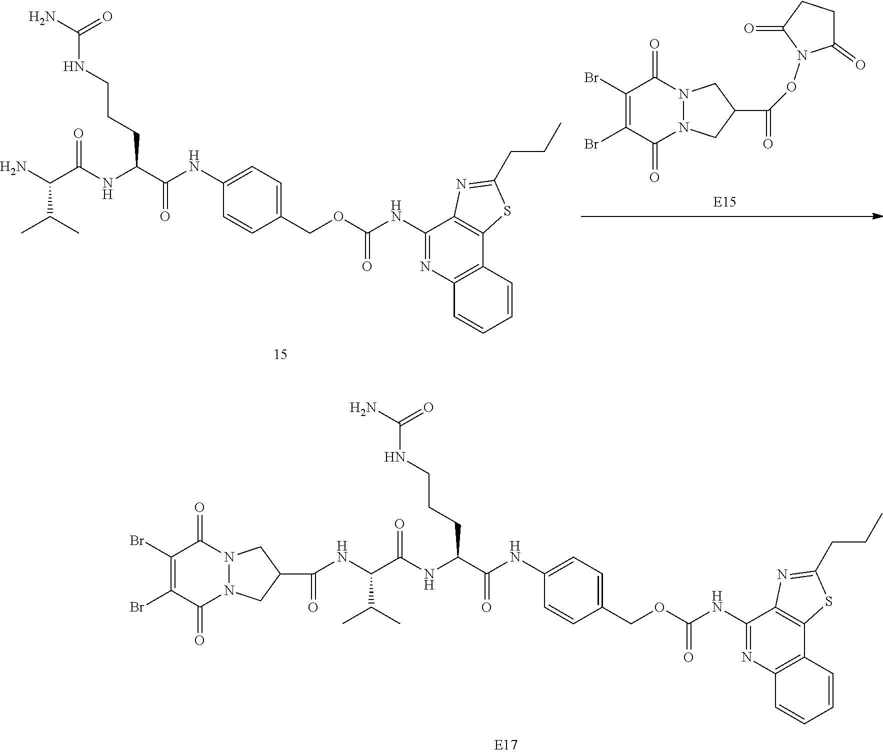

To a solution of compound 15 (15 mg) in DMF (1 mL) was added E15 (10 mg), followed by DIEA (8 mL). The reaction mixture was stirred at room temperature. After 3 h, the crude mixture was purified by RP-HPLC to give compound E17 as a white solid (13 mg) after lyophilization. MS found: 985.2 [M+H⁺].

### Example 26

### Synthesis of (14S,33S,2R,4S,10E,12E,14R)-86-chloro-14-hydroxy-85,14-dimethoxy-33,2,7,10-tetramethyl-12,6-dioxo-7-aza-1(6,4)-oxazinana-3(2,3)-oxirana-8(1,3)-benzenacyclotetradecaphane-10,12-dien-4-yl N-(6-(6,7-dibromo-5,8-dioxo-2,3,5,8-tetrahydro-1H-pyrazolo[1,2-a]pyridazine-2-carboxamido)hexanoyl)-N-methyl-L-alaninate (18)

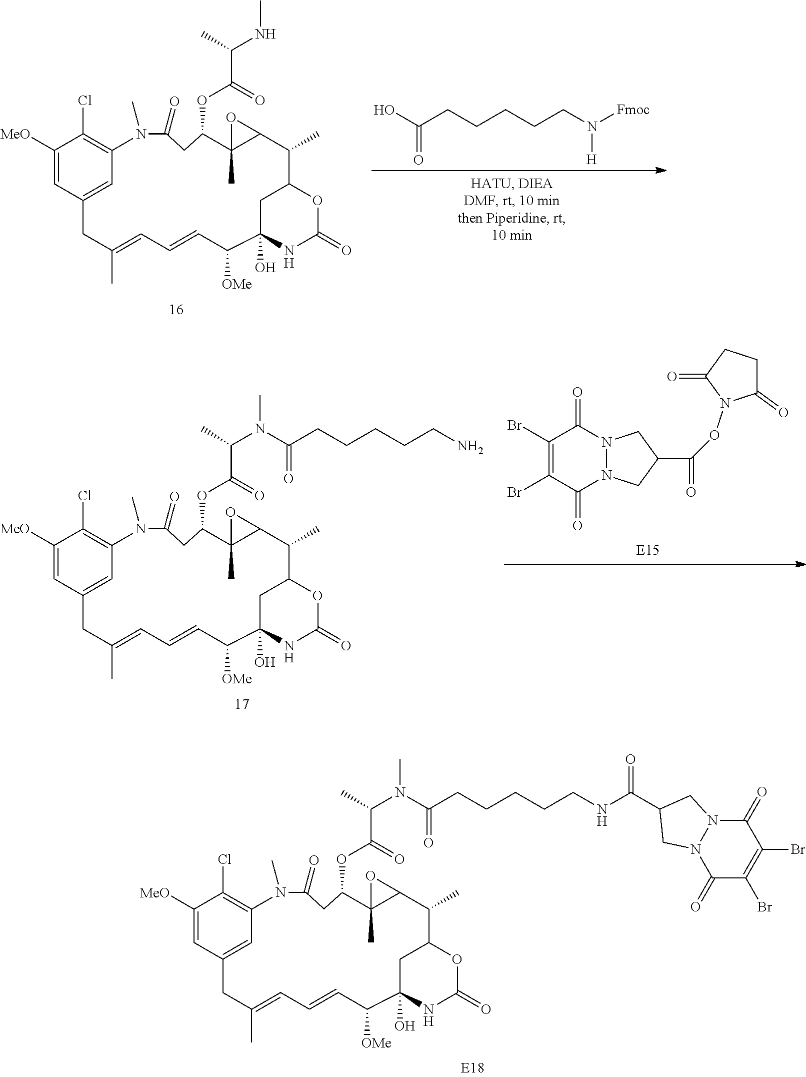

To a solution of compound 16 (65 mg) in anhydrous DMF (2 mL) was added Fmoc-aminohexanoic acid (38 mg) followed by DIEA (40 mL) and HATU (42 mg). The reaction mixture was stirred at room temperature (22° C.). After 10 min, piperidine (100 mL) was added and the reaction was stirred at room temperature for 30 min. The mixture was purified directly by RP-HPLC to give compound 17 as a white solid (72 mg, TFA salt) after lyophilization.

To a solution of compound 17 (17 mg) in DMF (1 mL) was added E15 (10 mg), followed by DIEA (7 mL). The reaction mixture was stirred at room temperature. After 15 minutes, the crude mixture was purified by RP-HPLC to give compound E18 as a white solid (16 mg) after lyophilization. MS found: 1099.4 [M+H⁺].

### Example 27

### Synthesis of 4-((S)-2-((S)-2-(6,7-dibromo-5,8-dioxo-2,3,5,8-tetrahydro-1H-pyrazolo[1,2-a]pyridazine-2-carboxamido)propanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (E19)

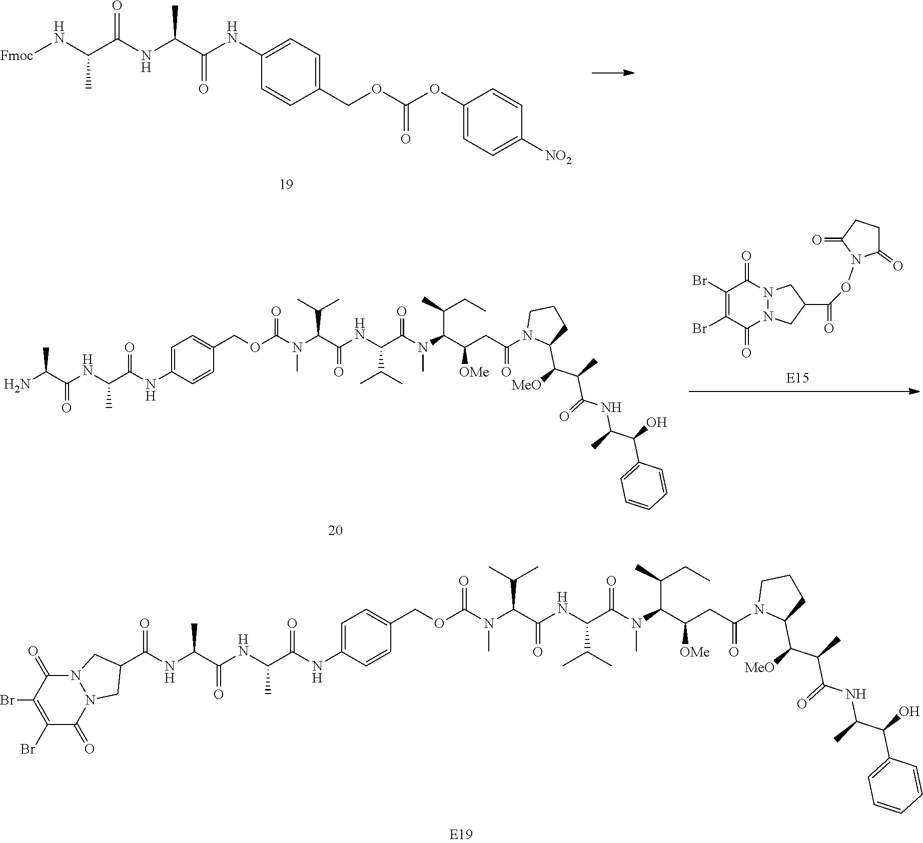

To a solution of compound 19 (35 mg) in anhydrous DMF (1 mL) was added monomethyl Auristatin E (35 mg), followed by DIEA (10 mL) and HOBt (2 mg). The reaction mixture was stirred at room temperature (22° C.). After 24 h, piperidine (100 mL) was added and the reaction was stirred at room temperature for 15 minutes. The mixture was purified directly by RP-HPLC to give compound 20 as a white solid (43 mg, TFA salt) after lyophilization.

To a solution of compound 20 (11 mg) in DMF (1 mL) was added E15 (6 mg), followed by DIEA (5 mL). The reaction mixture was stirred at room temperature. After 1 h, the crude mixture was purified by RP-HPLC to give compound E19 as a white solid (10 mg) after lyophilization. MS found: 1345.6 [M+H⁺].

### Example 28

### 4-((S)-2-((S)-2-(6,7-dibromo-5,8-dioxo-2,3,5,8-tetrahydro-1H-pyrazolo[1,2-a]pyridazine-2-carboxamido)-3-methylbutanamido)propanamido)benzyl ((S)-1-(((S)-1-(((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxoheptan-4-yl)(methyl)amino)-3-methyl-1-oxobutan-2-yl)amino)-3-methyl-1-oxobutan-2-yl)(methyl)carbamate (E20)

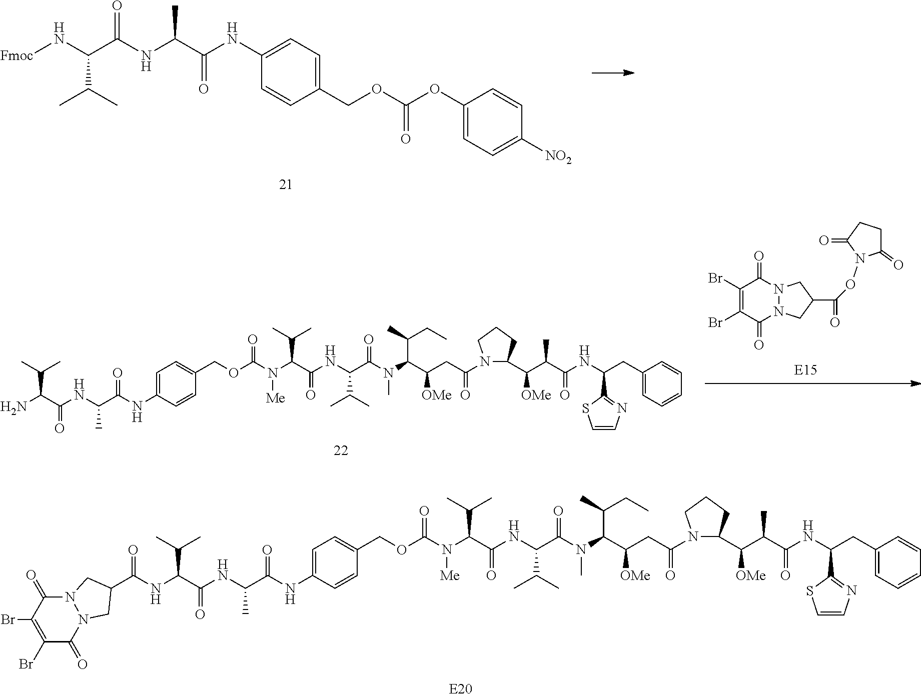

To a solution of compound 21 (39 mg) in anhydrous DMF (1 mL) was added monomethyl Dolastatin 10 (38 mg), followed by DIEA (10 mL) and HOBt (2 mg). The reaction mixture was stirred at room temperature (22° C.). After 24 h, piperidine (100 mL) was added and the reaction was stirred at room temperature for 15 minutes. The mixture was purified directly by RP-HPLC to give compound 22 as a white solid (49 mg, TFA salt) after lyophilization.

To a solution of compound 22 (12 mg) in DMF (1 mL) was added E15 (6 mg), followed by DIEA (5 mL). The reaction mixture was stirred at room temperature. After 3 h, the crude mixture was purified by RP-HPLC to give compound E20 as a white solid (11 mg) after lyophilization. MS found: 1426.6 [M+H⁺].

### Example 29

### Synthesis of ((2R,3R)-3-((S)-1-((3R,4S,5S)-4-((S)-2-((S)-2-((((4-((S)-2-((S)-2-(6,7-dibromo-5,8-dioxo-2,3,5,8-tetrahydro-1H-pyrazolo[1,2-a]pyridazine-2-carboxamido)-3-methylbutanamido)-5-ureidopentanamido)benzyl)oxy)carbonyl)(methyl)amino)-3-methylbutanamido)-N,3-dimethylbutanamido)-3-methoxy-5-methylheptanoyl)pyrrolidin-2-yl)-3-methoxy-2-methylpropanoyl)-L-phenylalanine (E21)

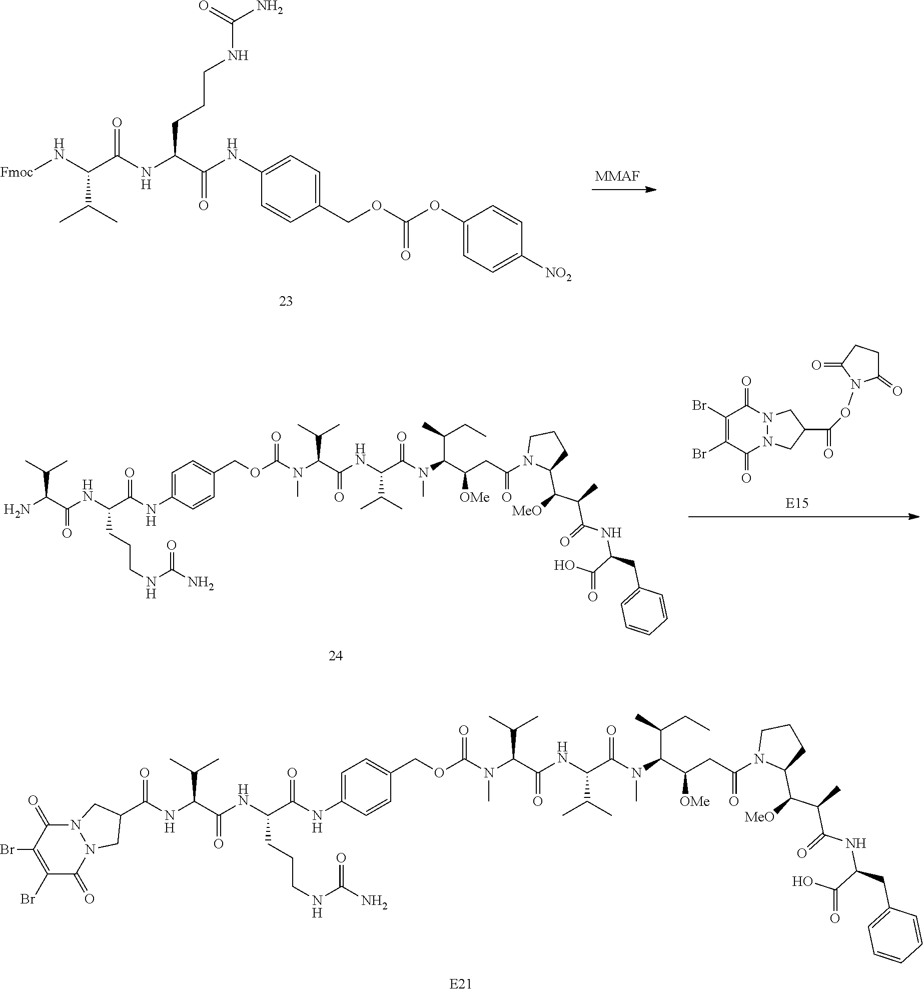

To a solution of compound 23 (43 mg) in anhydrous DMF (2 mL) was added monomethyl Auristatin F (TFA salt, 41 mg), followed by DIEA (15 mL) and HOBt (2 mg). The reaction mixture was stirred at room temperature (22° C.). After 24 h, piperidine (100 mL) was added and the reaction was stirred at room temperature for 15 minutes. The mixture was purified directly by RP-HPLC to give compound 24 as a white solid (47 mg, TFA salt) after lyophilization.

To a solution of compound 24 (12 mg) in DMF (1 mL) was added E15 (6 mg), followed by DIEA (5 mL). The reaction mixture was stirred at room temperature. After 3 h, the crude mixture was purified by RP-HPLC to give compound E21 as a white solid (10 mg) after lyophilization. MS found: 1473.8 [M+H⁺].

### Example 30

### Synthesis of 6,7-dibromo-N-(2-((2-(((S)-1-((2-(((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl)amino)-1-oxo-3-phenylpropan-2-yl)amino)-2-oxoethyl)amino)-2-oxoethyl)-5,8-dioxo-2,3,5,8-tetrahydro-TH-pyrazolo[1,2-a]pyridazine-2-carboxamide (E22)

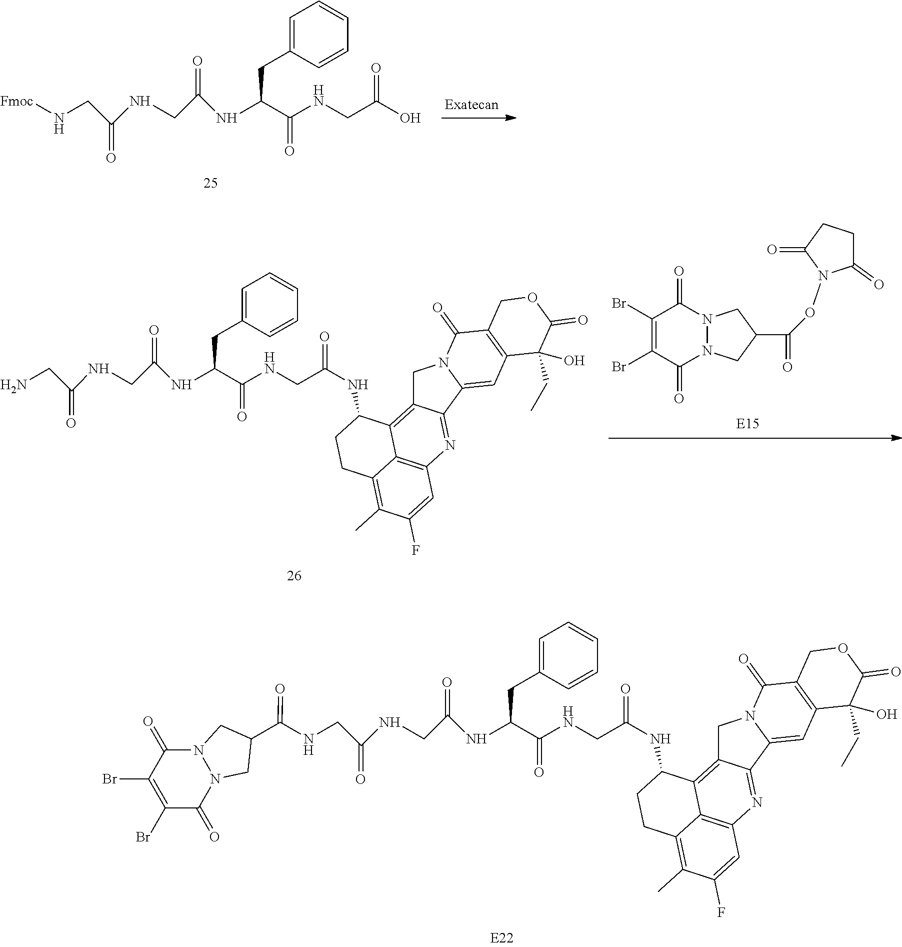

To a suspension of Exatecan mesylate (53 mg) in anhydrous DMF (2 mL) was added Fmoc-Gly-Gly-Phe-Gly-OH (compound 25, 62 mg) followed by DIEA (54 mL) and PyAOP (60 mg). The reaction mixture was stirred at room temperature (22° C.). After 10 min, piperidine (100 mL) was added and the reaction was stirred at room temperature for 30 min. The mixture was purified directly by RP-HPLC to give compound 26 as a yellow solid (62 mg, TFA salt) after lyophilization.

To a solution of compound 26 (17 mg) in DMF (1 mL) was added E15 (10 mg), followed by DIEA (7 mL). The reaction mixture was stirred at room temperature. After 10 minutes, the crude mixture was purified by RP-HPLC to give compound E22 as a yellow solid (14 mg) after lyophilization. MS found: 1090.3 [M+H⁺].

### Example 31

### Synthesis of 4-((S)-2-(6,7-dibromo-5,8-dioxo-2,3,5,8-tetrahydro-1H-pyrazolo[1,2-a]pyridazine-2-carboxamido)propanamido)benzyl ((S)-4,11-diethyl-9-hydroxy-3,14-dioxo-3,4,12,14-tetrahydro-1H-pyrano[3′,4′:6,7]indolizino[1,2-b]quinolin-4-yl) carbonate (E23)

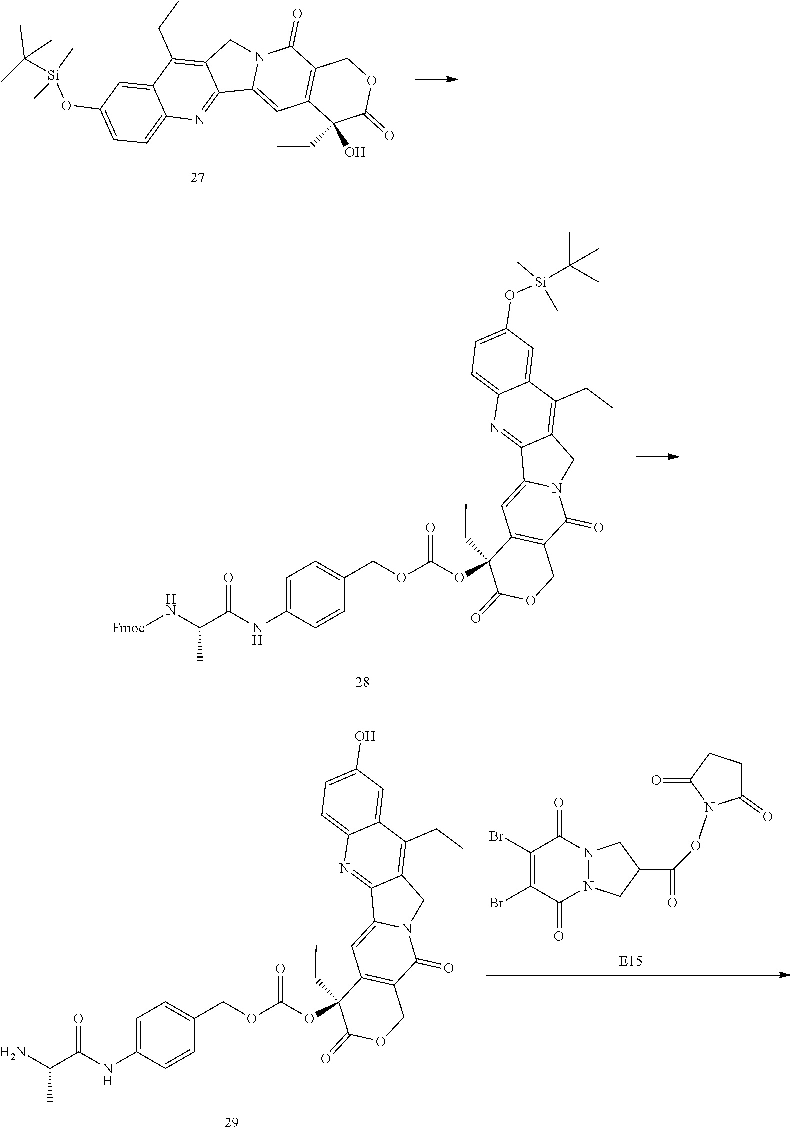

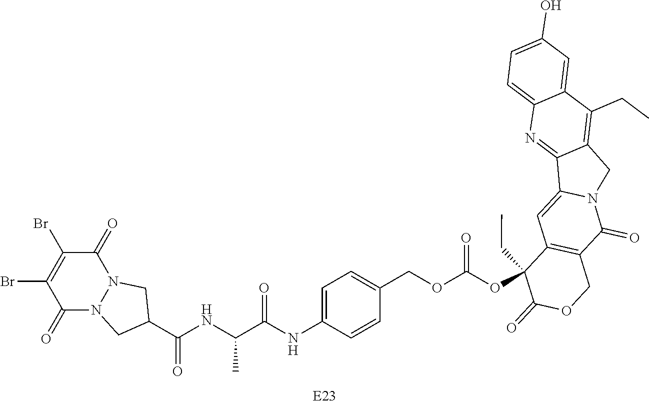 [SMILES: CCc1c2c(nc3ccc(O)cc13)-c1cc3c(c(=O)n1C2)COC(=O)[C@@]3(CC)OC(=O)OCc1ccc(NC(=O)[C@H](C)NC(=O)C2Cn3c(=O)c(Br)c(Br)c(=O)n3C2)cc1]

To a solution of 10-TBDMS-SN38 (compound 27, 50 mg) in anhydrous DCM (2 mL) was added triphosgene (15 mg), followed by DMAP (60 mg). The mixture was stirred at room temperature for 5 min, then Fmoc-Ala-PAB-OH (42 mg) was added. The reaction was kept at room temperature for 30 min. The crude reaction was diluted with DCM (30 mL) and washed with water (20 mL). The organic layer was concentrated to dryness and the residue (compound 28) was redissolved in DMF (2 mL). Piperidine (100 mL) was added, and after 15 min, TBAF (1 M in THF, 0.2 mL) was added. The reaction was stirred at room temperature for 15 min and the crude product was purified directly by RP-HPLC to give compound 29 as a yellow solid (34 mg) after lyophilization.

To a solution of compound 29 (14 mg) in DMF (1 mL) was added E15 (10 mg), followed by DIEA (7 mL). The reaction mixture was stirred at room temperature. After 30 minutes, the crude mixture was purified by RP-HPLC to give compound E23 as a yellow solid (12 mg) after lyophilization. MS found: 949.2 [M+H⁺].

### Example 32

### Synthesis of 6-mono bromo-5,8-dioxo-2,3,5,8-tetrahydro-1H-pyrazolo[1,2-a]pyridazine-2-carboxylic acid (E24)

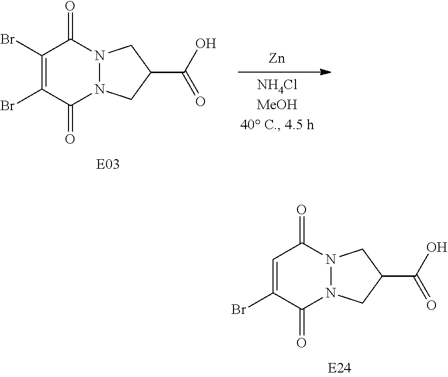

6,7-dibromo-5,8-dioxo-2,3,5,8-tetrahydro-1H-pyrazolo[1,2-a]pyridazine-2-carboxylic acid (0.50 g, 1.4 mmol) was dissolved in methanol (5 mL) and treated with ammonium chloride (0.21 g, 2.9 eq), followed by Zn powder (0.27 g, 3.0 eq) with stirring. The mixture was heated at 40 C for 4.5 h, then quenched with NH₄Cl aq. and extracted with dichloromethane (15 mL×3). The combined extracts were washed with brine, dried with Na₂SO₄, filtered, and evaporated to the dryness. The residue was purified by a SiO₂ pad to afford 0.23 g of 6-bromo-5,8-dioxo-2,3,5,8-tetrahydro-1H-pyrazolo[1,2-a]pyridazine-2-carboxylic acid (80% pure by HPLC) as an off-white solid (yield 60%). This product was used without further purification. MS (M+H): 275.0, 277.1.

It is understood that the examples and embodiments described herein are for illustrative purposes only and that various modifications or changes in light thereof will be suggested to persons skilled in the art and are to be included within the spirit and purview of this application and scope of the appended claims. All patent or non-patent references mentioned herein are incorporated by reference in their entireties without admission of them as prior art.

1. A compound of formula (I): 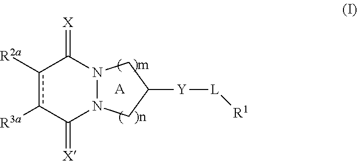
or a salt thereof, wherein:
Ring A is a 5 to 13-membered heterocycle;
m and n are each independently 1, 2, 3, 4, or 5; [symbol]
represents a double or single bond;
X and X′ are each independently O, S, or NR^{x}, wherein R^{x} is C1-C6 alkyl, C2-C6 alkenyl, or C2-C6 alkynyl;
Y is a bond, (CH₂)ᵢ (i=integer from 1 to 12), C(O), C(O)O, OC(O), C(O)NR^{a}, NR^{a}C(O), NR^{a}, O, S, S(O), S(O)₂, substituted or unsubstituted C6-C10 arylene, or substituted or unsubstituted 5 to 12-membered heteroarylene, or a combination thereof,
R^{2a} and R^{3a} are each independently halogen, —U—R², or —V—R³;
U and V are independently S, S(O), S(O)₂, O, or NH;
L is a bond or linear or branched-chain linker comprising a group or moiety selected from substituted or unsubstituted alkylene, substituted or unsubstituted cyclylene, substituted or unsubstituted heterocyclylene, substituted or unsubstituted arylene, substituted or unsubstituted heteroarylene, O, S, NR^{a}, C(O), C(O)O, OC(O), C(O)NH, NHC(O), S(O), S(O)₂, (CH₂CH₂O)ⱼ, (OCH₂CH₂)ⱼ (PEGn, j=2 to 48), S—S, hydrazone, substituted or unsubstituted oligo peptide (e.g., Val-Cit, Gly-Gly-Phe-Gly, Val-Ala, Ala-Ala, Ala-Ala-Asn, Phe-Lys, Val-Lys, or Val-Arg), and combinations thereof, wherein R^{a} is hydrogen or C₁-C₆ alkyl, and wherein the linker L optionally comprises a self-immolative spacer;
R¹ is substituted or unsubstituted alkyl, substituted or unsubstituted cycloalkyl, substituted or unsubstituted aryl, substituted or unsubstituted heteroaryl, substituted or unsubstituted heterocyclyl, substituted or unsubstituted succinimidyl, or a functional moiety selected from: a leaving group (e.g., BSA, KLH, and OVA), a detectable moiety, an enzymatically active moiety, an affinity tag, a hapten, an immunogenic carrier, radionuclides, photosensitizers, cytotoxins and their prodrugs, innate immune modulators, biopolymers, oligonucleotides, PROTAC degraders, antibiotics, and exotoxin;
R² and R³ are each independently selected from substituted or unsubstituted alkyl, substituted or unsubstituted alkenyl, substituted or unsubstituted alkynyl, substituted or unsubstituted aryl, substituted or unsubstituted heteroaryl, substituted or unsubstituted cycloalkyl, substituted or unsubstituted heterocyclyl, antibody, antigen, liposome, polymeric moiety, substituted or unsubstituted amino acid moiety, substituted or unsubstituted peptide moiety, DNA, RNA, virus or virus-like particles, and targeting ligand small molecule which carries a nucleophilic group or molecular moiety, such as —SH, —OH, —NH₂, guanidinyl, imidazolyl, indolyl, and carboxylic acid (—CO₂H);
or alternatively, R² and R³ together become R⁴, which together with U, V, and [symbol] forms a ring B as characterized in formula (II): 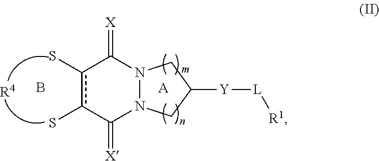
wherein R⁴ is a moiety of antibody, antigen, liposome, polymer, amino acid, peptide, DNA, RNA, virus, virus-like particles, or targeting ligand small molecule, wherein the targeting ligand small molecule optionally comprises a nucleophilic group selected from —SH, —OH, —NH₂, guanidinyl, imidazolyl, indolyl, and carboxylic acid, or a combination thereof.

2. The compound of claim 1, or a salt thereof, wherein:
Ring A is a 5 to 9-membered heterocycle; and
m and n are each independently 1, 2, or 3.

3. The compound of claim 1, or a salt thereof, wherein:
Ring A is a 5 to 7-membered heterocycle; and
m and n are each independently 1 or 2.

4. The compound of claim 1, or a salt thereof, wherein:
m=1, and n=1.

5. The compound of claim 1, or a salt thereof, wherein: [symbol]
is a double bond, and X and X′ are each O.

6. The compound of claim 1, or a salt thereof, wherein:
R^{2a} and R^{3a} are each halogen.

7. The compound of claim 6, or a salt thereof, wherein:
R^{2a} and R^{3a} are each bromo (Br).

8. The compound of claim 1, or a salt thereof, wherein:
U and V are each sulfur (S); and
R² and R³ are each independently alkyl, aryl, heteroaryl, cycloalkyl, heterocyclyl, or an amino acid moiety, each optionally substituted.

9. The compound of claim 1, or a salt thereof,
wherein:
U and V are each sulfur (S); and
R² and R³ are each independently or together selected from antibody, antigen, liposome, polymeric moiety, amino acid, oligopeptide, DNA, RNA, virus or virus-like particles, and targeting ligand small molecule.

10. The compound of claim 1, or a salt thereof,
wherein:
Y is C(O)O, C(O)NH, CH₂, O, S, or NH;
L is a bond, alkylene, or —(CH₂)ₖC(O)NH—, wherein k is an integer selected from 1 to 8; and
R¹ is alkyl, cycloalkyl, aryl, or succinimidyl.

11. The compound of claim 1, or a salt thereof, wherein:
Y is C(O)NH;
L is a bond, —(CH₂)ₖC(O)NH—, or oligopeptide moiety, or a combination thereof, wherein k is an integer selected from 1 to 8; and
R¹ is selected from alkyl, cycloalkyl, aryl, a detectable moiety, an enzymatically active moiety, an affinity tag, a hapten, an immunogenic carrier, radionuclides, photosensitizers, cytotoxins and their prodrugs, innate immune modulators, biopolymers, oligonucleotides, PROTAC degraders, antibiotics, and exotoxin.

12. The compound of claim 11, or a salt thereof, wherein the oligopeptide moiety is selected from Val-Cit, Gly-Gly-Phe-Gly, Val-Ala, Ala-Ala, Ala-Ala-Asn, Phe-Lys, Val-Lys, and Val-Arg.

13. The compound of claim 1, wherein the self-immolative spacer comprises a para-aminobenzyloxycarbonyl (PABC) moiety or a PABC-type moiety (e.g., ortho-aminobenzyl, ortho-hydroxybenzyl, and para-hydroxybenzyl) that can lead to electron cascade-mediated self-immolation, or a cyclization-mediated self-immolation.

14. A compound of Formula (II): 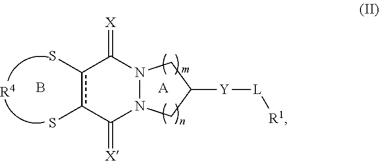
or a pharmaceutically acceptable salt thereof, wherein
Ring A is independently 5 to 13-membered carbocycles;
m and n are each independently 1, 2, 3, 4, or 5; Ring B comprises two nonnucleophilic groups (e.g., thiol groups derived from a disulfide bridge in a peptide, protein, or antibody;
X and X′ are each independently O, S, or NR^{x};
Y is a bond, (CH₂)ᵢ (i=integer from 1 to 12), C(O), C(O)O, NR^{a}, O, S, S(O), S(O)₂, substituted or unsubstituted C6-C10 arylene, or substituted or unsubstituted 5 to 12 membered heteroarylene, or a combination thereof;
L is a bond or linear or branched-chain linker comprising a group or moiety selected from alkylene, O, S, NR^{a}, C(O), C(O)O, OC(O), C(O)NH, NHC(O), S(O), S(O)₂, (CH₂CH₂O)ⱼ, (OCH₂CH₂)ⱼ (PEGn, j=2 to 48), S—S, hydrazone, oligo peptide, (e.g., Val-Cit, Gly-Gly-Phe-Gly, Val-Ala, Ala-Ala, Ala-Ala-Asn, Phe-Lys, Val-Lys, or Val-Arg), and combinations thereof, wherein the linker optionally comprises a self-immolative spacer;
R¹ is alkyl, cycloalkyl, aryl, succinimidyl, or a functional moiety selected from: a leaving group, a detectable moiety, an enzymatically active moiety, an affinity tag, a hapten, an immunogenic carrier, radionuclides, photosensitizers, cytotoxins and their prodrugs, innate immune modulators, biopolymers, oligonucleotides, PROTAC degraders, antibiotics, and exotoxin.
R^{x} is C1-C6 alkyl, C2-C6 alkenyl, or C2-C6 alkynyl;
R⁴ is a moiety of antibody, antigen, liposome, polymer, amino acid, peptide, DNA, RNA, virus, virus-like particles, or targeting ligand small molecule, wherein the targeting ligand small molecule optionally comprises a nucleophilic group selected from —SH, —OH, —NH₂, guanidine, imidazole, indole, carboxylic acid, or a combination thereof, and
R^{a} is hydrogen or C₁-C₆ alkyl.

15. The compound of claim 14, or a salt thereof, wherein:
Ring A is a 5 to 9-membered heterocycle; and
m and n are each independently 1, 2, or 3.

16. The compound of claim 14, or a salt thereof, wherein:
Ring A is a 5 to 7-membered heterocycle; and
m and n are each independently 1 or 2.

17. The compound of claim 14, or a salt thereof, wherein:
m=1, and n=1.

18. The compound of claim 14, or a salt thereof, wherein: [symbol]
is a double bond, and X and X′ are each O.

19. (canceled)

20. (canceled)

21. (canceled)

22. (canceled)

23. The compound of claim 14, or a salt thereof, wherein:
Y is C(O)O, C(O)NH, CH₂, O, S, or NH;
L is a bond, alkylene, or —(CH₂)ₖC(O)NH— (k is an integer selected from 1 to 8); and
R¹ is alkyl, cycloalkyl, aryl, or succinimidyl.

24. The compound of claim 14, or a salt thereof, wherein:
Y is C(O)NH;
L is a bond, —(CH₂)ₖC(O)NH— (k is an integer selected from 1 to 8), or oligopeptide moiety, or a combination thereof, and
R¹ is selected from alkyl, cycloalkyl, aryl, a detectable moiety, an enzymatically active moiety, an affinity tag, a hapten, an immunogenic carrier, radionuclides, photosensitizers, cytotoxins and their prodrugs, innate immune modulators, biopolymers, oligonucleotides, PROTAC degraders, antibiotics, and exotoxin.

25. The compound of claim 24, or a salt thereof, wherein the oligopeptide moiety is selected from Val-Cit, Gly-Gly-Phe-Gly, Val-Ala, Ala-Ala, Ala-Ala-Asn, Phe-Lys, Val-Lys, and Val-Arg.

26. The compound of claim 14, wherein the self-immolative spacer comprises a para-aminobenzyloxycarbonyl (PABC) moiety or a PABC-type moiety (e.g., ortho-aminobenzyl, ortho-hydroxybenzyl, and para-hydroxybenzyl) that can lead to electron cascade-mediated self-immolation, or a cyclization-mediated self-immolation.

27. A compound selected from compounds E1 through E15 and E17 through E24, or a salt or stereoisomer thereof:

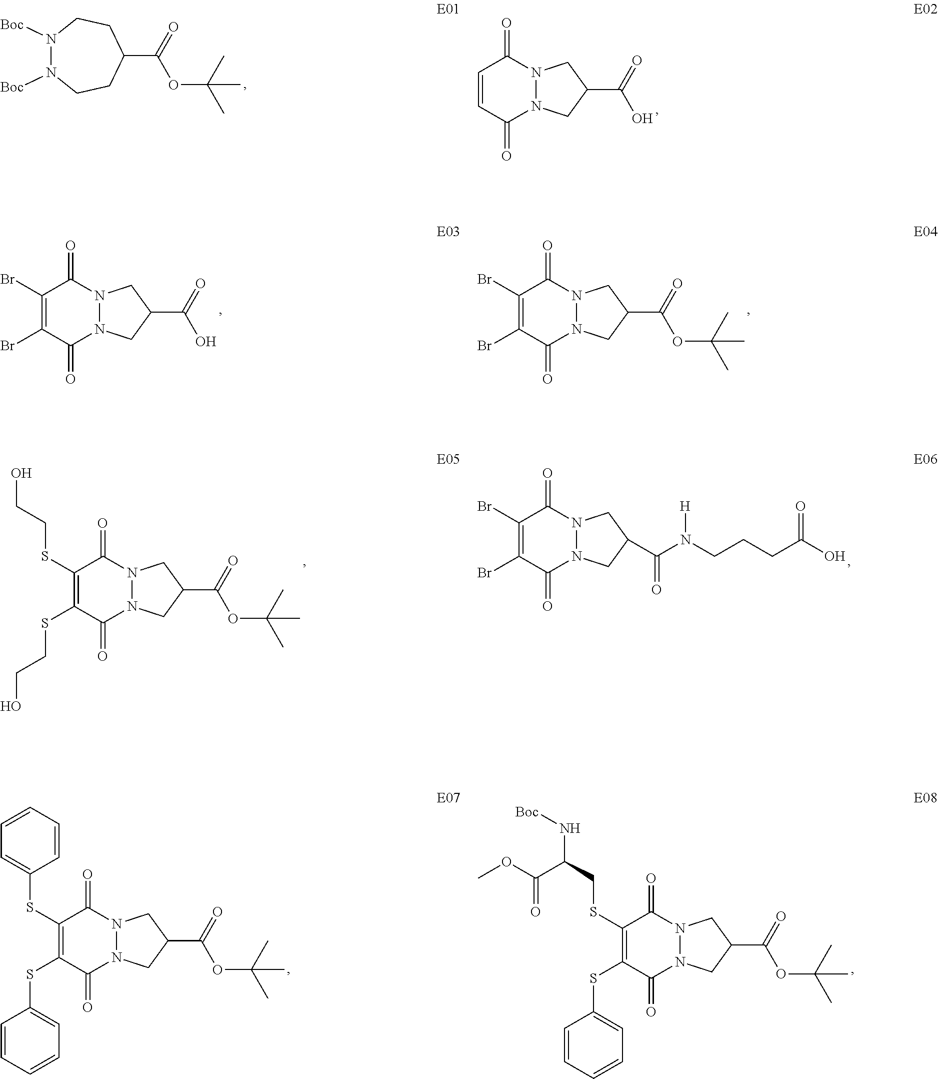

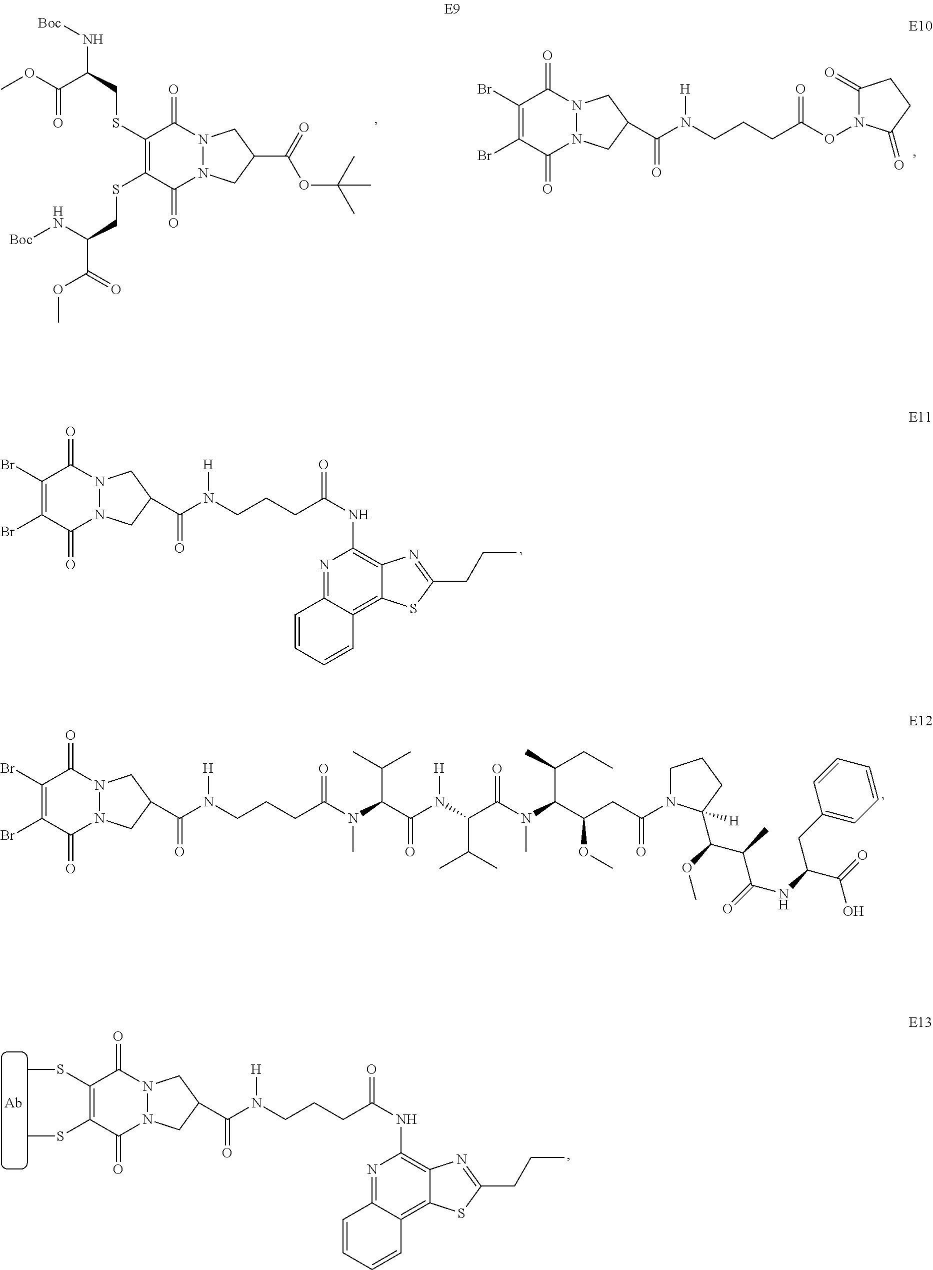 [SMILES: COC(=O)[C@H](CSc1c(SC[C@H](NC(=O)OC(C)(C)C)C(=O)OC)c(=O)n2n(c1=O)CC(C(=O)OC(C)(C)C)C2)NC(=O)OC(C)(C)C ; O=C(CCCNC(=O)C1Cn2c(=O)c(Br)c(Br)c(=O)n2C1)ON1C(=O)CCC1=O ; CCCc1nc2c(NC(=O)CCCNC(=O)C3Cn4c(=O)c(Br)c(Br)c(=O)n4C3)nc3ccccc3c2s1 ; CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCNC(=O)C1Cn2c(=O)c(Br)c(Br)c(=O)n2C1)C(C)C ; CCCc1nc2c(NC(=O)CCCNC(=O)C3Cn4c(=O)c(SC)c(SC)c(=O)n4C3)nc3ccccc3c2s1]

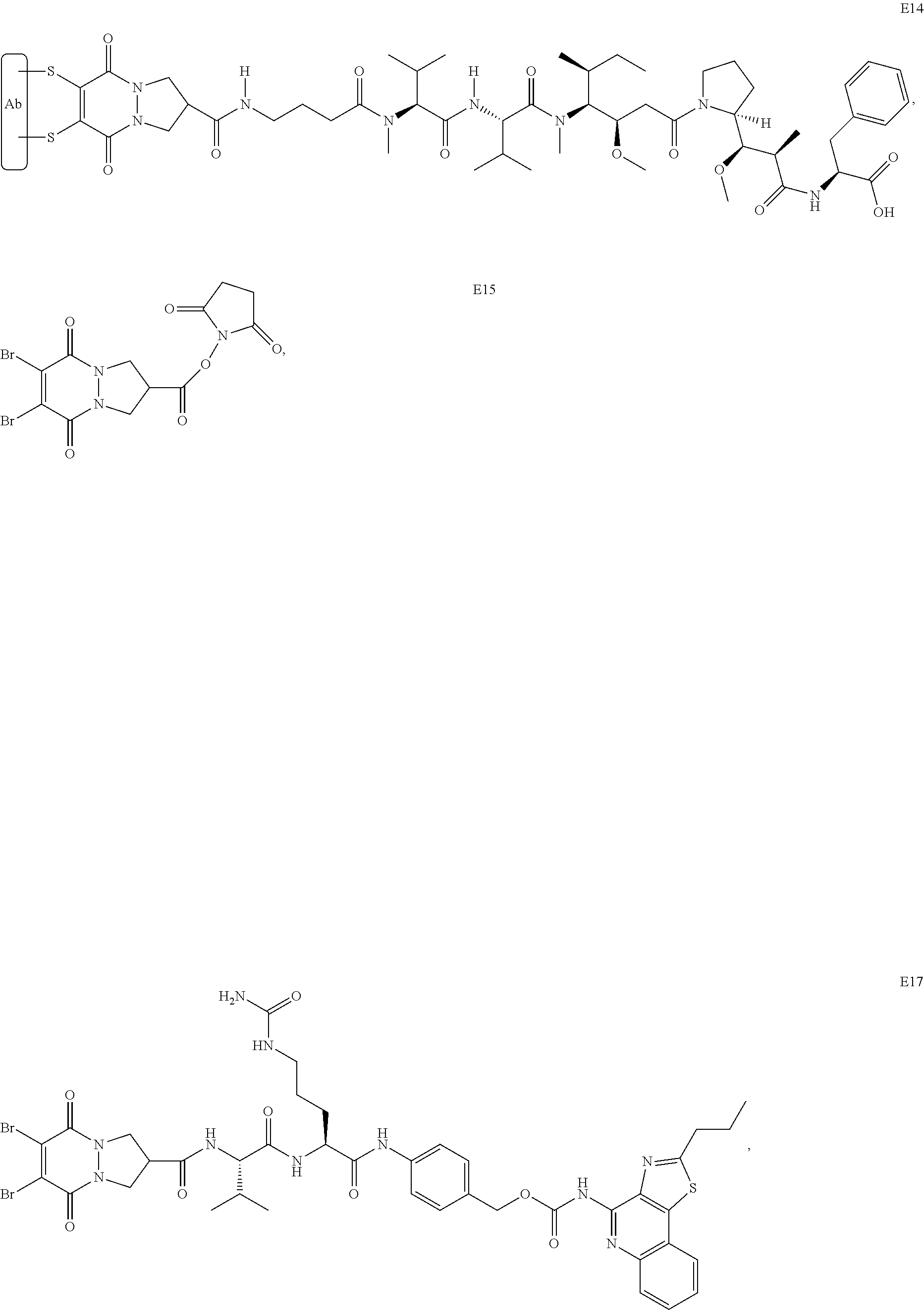 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCNC(=O)C1Cn2c(=O)c(SC)c(SC)c(=O)n2C1)C(C)C ; O=C(ON1C(=O)CCC1=O)C1Cn2c(=O)c(Br)c(Br)c(=O)n2C1 ; CCCc1nc2c(NC(=O)OCc3ccc(NC(=O)[C@H](CCCNC(N)=O)NC(=O)[C@@H](NC(=O)C4Cn5c(=O)c(Br)c(Br)c(=O)n5C4)C(C)C)cc3)nc3ccccc3c2s1]

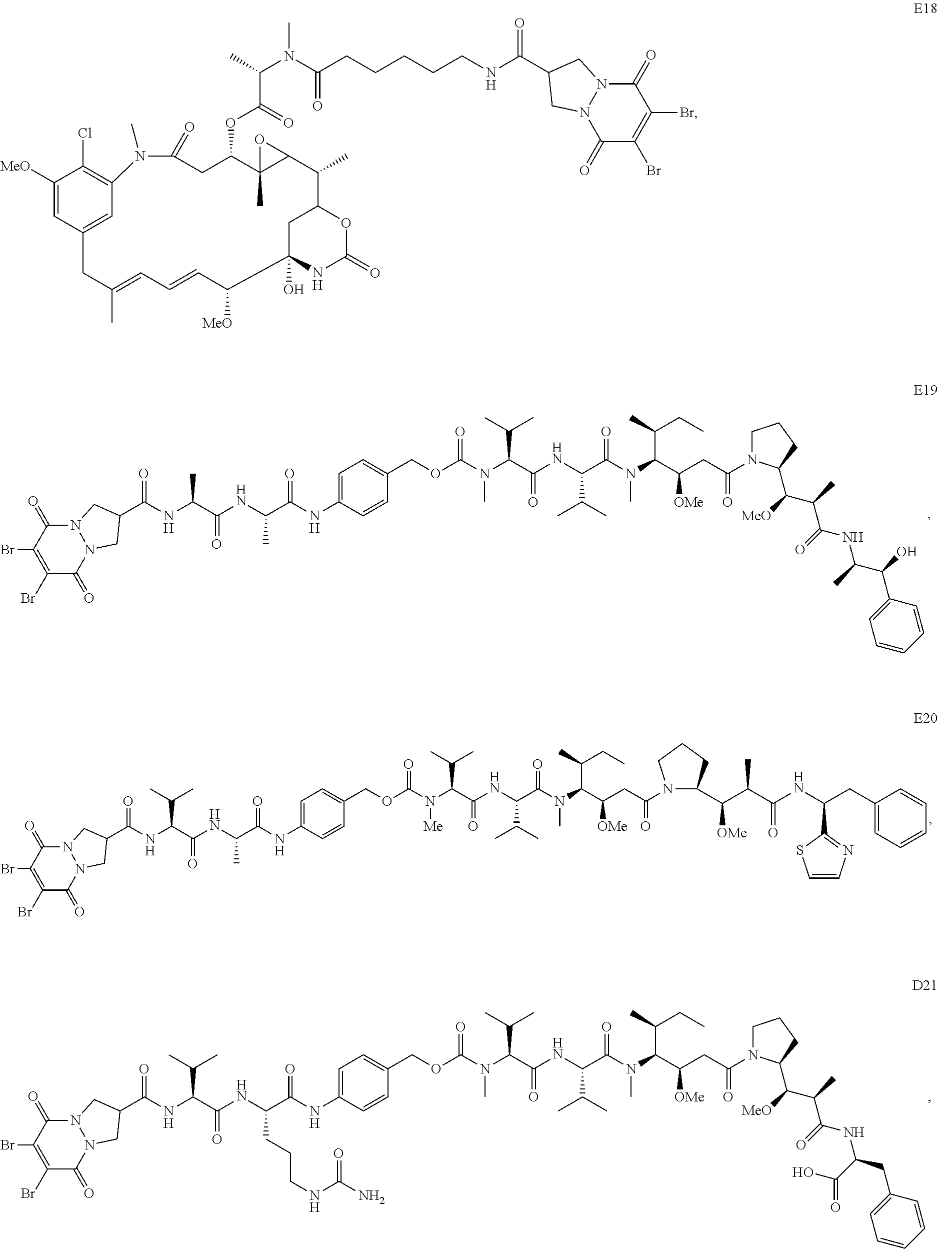

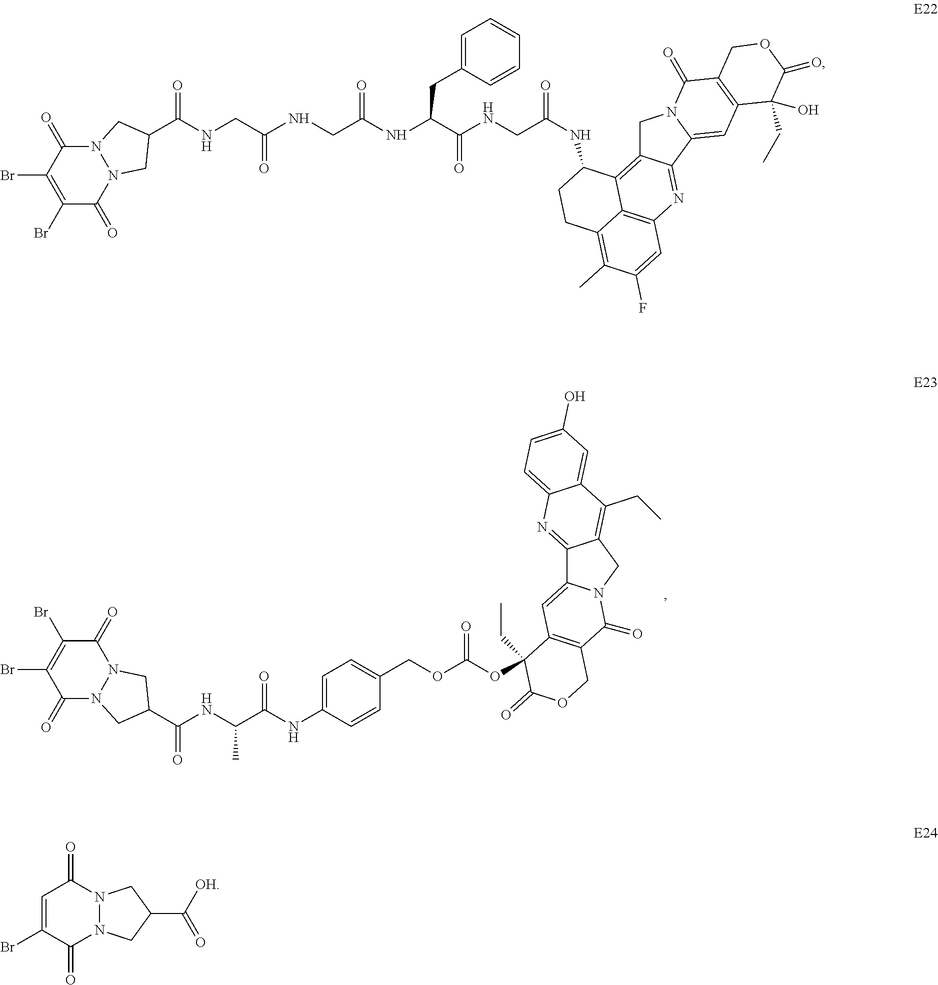 [SMILES: CC[C@@]1(O)C(=O)OCc2c1cc1n(c2=O)Cc2c-1nc1cc(F)c(C)c3c1c2[C@@H](NC(=O)CNC(=O)[C@H](Cc1ccccc1)NC(=O)CNC(=O)CNC(=O)C1Cn2c(=O)c(Br)c(Br)c(=O)n2C1)CC3 ; CCc1c2c(nc3ccc(O)cc13)-c1cc3c(c(=O)n1C2)COC(=O)[C@@]3(CC)OC(=O)OCc1ccc(NC(=O)[C@H](C)NC(=O)C2Cn3c(=O)c(Br)c(Br)c(=O)n3C2)cc1 ; O=C(O)C1Cn2c(=O)cc(Br)c(=O)n2C1]

28. A pharmaceutical composition comprising a compound according to claim 1 and a pharmaceutically acceptable carrier.

29. A method of treating a disease or disorder, comprising administering to a subject in need thereof a compound according to claim 1, or a pharmaceutically acceptable salt thereof.

30. (canceled)
